(19) United States
(12) Patent Application Publication
(10) Pub. No.: US 2024/0055114 A1
(43) Pub. Date: Feb. 15, 2024

(54) DRUG ADMINISTRATION DEVICE AND SYSTEM FOR ESTABLISHING A DOSAGE REGIMEN AND COMPATIBILITY OF COMPONENTS

(71) Applicant: Janssen Pharmaceuticals, lnc., Titusville, NJ (US)

(72) Inventors: Francesco N. Albertini, Pleasanton, CA (US); Gregory J. Bakos, Mason, OH (US); Jason L. Harris, Lebanon, OH (US); Emma Louise Hubert, San Jose, CA (US); Monica A. Kapil, San Jose, CA (US); Peter Krulevitch, Pleasanton, CA (US); David Ramos, Jacksonville, FL (US); Frederick E. Shelton, Hillsboro, OH (US); Jingli Wang, San Jose, CA (US)

(21) Appl. No.: 17/641,364

(22) PCT Filed: Sep. 24, 2020
(86) PCT No.: PCT/IB2020/058958

§ 371 Date: Mar. 8, 2022

Related U.S. Application Data
(60) Provisional application No. 63/020,935, filed on May 6, 2020.
(60) Provisional application No. 62/905,452, filed on Sep. 25, 2019.
(60) Provisional application No. 62/905,453, filed on Sep. 25, 2019.

(51) Int. Cl. G16H 40/63; G16H 20/17; A61M 5/20; G16H 20/13; A61M 5/14; A61M 15/00; A61M 11/00; A61M 99/00

(52) U.S. Cl. CPC G16H 40/63; G16H 20/17; A61M 5/2033; G16H 20/13; A61M 5/14; A61M 15/0045; A61M 11/007; A61M 99/00; A61M 2005/14208

(57) ABSTRACT

The present disclosure provides methods, systems, and devices for establishing dosing parameters for a drug administration device and establishing compatibility of components of the drug administration device. In an exemplary embodiment, a method can include acquiring data from a drug holder and communicating the data to a processor, communicating a relevant subset of dosing parameter data from a server to the processor, and establishing dosing parameters for a drug administration device based on the relevant subset of dosing parameter data. The relevant subset of dosing parameter data is determined based on the drug holder data. The method can include acquiring first component data relating to a first component of the drug administration device, comparing the first component data with acceptable first component data, and setting an operational status of the drug administration device based on the comparison.

### CROSS REFERENCE TO RELATED APPLICATIONS

The present application claims priority to Intl. Pat. App. No. PCT/IB2020/058958 entitled “Drug Administration Device And System For Establishing A Dosage Regimen And Compatibility Of Components” filed Sep. 24, 2020, which claims priority to U.S. Prov. Pat. App. 63/020,935 entitled “Drug Administration Device And System For Establishing A Dosage Regimen And Compatibility Of Components” filed May 6, 2020, U.S. Prov. Pat. App. 62/905,453 entitled “Drug Administration Device And System For Establishing A Dosage Regimen And Compatibility Of Components” filed Sep. 25, 2019, and U.S. Prov. Pat. App. 62/905,452 entitled “Drug Administration Device And System For Establishing A Dosage Regimen And Compatibility Of Components” filed Sep. 25, 2019, which are hereby incorporated by reference in their entireties.

### FIELD

The embodiments described herein relate to a device for administering and/or provision of a drug. The present disclosure further relates to a system in which the device can be used, and a method of administration, and a further method associated with the system.

### BACKGROUND

Pharmaceutical products (including large and small molecule pharmaceuticals, hereinafter “drugs”) are administered to patients in a variety of different ways for the treatment of specific medical indications. Regardless of the manner of the administration, care must be taken when administering drugs to avoid adverse effects on the patient. For example, care must be taken not to administer more than a safe amount of the drug to the patient. This requires consideration of the amount of dose given and the time frame over which the dose is delivered, sometimes in relation to previous doses, or doses of other drugs. Moreover, care must be taken not to inadvertently administer an incorrect drug to the patient, or drugs that have degraded due to their age or storage conditions. All of these considerations can be conveyed in guidance associated with the specific drugs or drug combinations. However, this guidance is not always followed correctly, for example due to mistakes, such as human error. This can lead to adverse effects on the patient or result in inappropriate drug administration, for example insufficient or excessive volume of drug being administered for the specific medical indication.

Further, when it is possible to utilize different drugs in a given drug administration device or system, there is an increased risk that an incorrect dosage regimen will be used for the drug that is actually being delivered to a patient.

In relation to how a drug is administered to the patient, there are various dosage forms that can be used. For example, these dosage forms may include parenteral, inhalational, oral, ophthalmic, nasal, topical, and suppository forms of one or more drugs.

The dosage forms can be administered directly to the patient via a drug administration device. There are a number of different types of drug administration devices commonly available for delivery of the various dosage forms including: syringes, injection devices (e.g., autoinjectors Jet injectors, and infusion pumps), nasal spray devices, and inhalers.

Drug administration devices can be composed of a variety of different components. Inadvertent use of inappropriate components within the drug administration device can cause problems with the operation of the device and administration of the drug. It can result in an inappropriate dosing regimen being enacted, which could be detrimental to the health of the patient.

### SUMMARY

In one aspect, a method for establishing dosing parameters for a drug administration device is provided that in one embodiment includes acquiring drug holder data from a drug holder of the drug administration device and communicating the drug holder data to a processor. Either the drug administration device includes the processor, or an external device includes the processor. The method also includes communicating at least a relevant subset of dosing parameter data from a server to the processor, and establishing the dosing parameters for the drug administration device based on the relevant subset of dosing parameter data. The relevant subset of dosing parameter data is determined based on the drug holder data.

The method can vary in any number of ways. For example, the method can include communicating the drug holder data from the processor to the server, and determining the relevant subset of dosing parameter data on the server. In at least some embodiments, the method can also include communicating drug administration device data from the processor to the server, and the relevant subset of dosing parameter data can be determined based on both the drug holder data and the drug administration device data. The drug administration device data can differentiate the drug administration device from at least one other form of drug administration device.

For another example, the method can include determining the relevant subset of dosing parameter data using the processor after the communicating, and the communicating can include communicating dosing parameter data including the relevant subset of dosing parameter data and a non-relevant subset of dosing parameter data. In at least some embodiments, the relevant subset of dosing parameter data can be determined based on both the drug holder data and drug administration device data. The drug administration device data can differentiate the drug administration device from at least one other form of drug administration device.

For yet another example, the communication between the processor and the server can include encrypted communication. For still another example, communication between the drug holder and the processor can include encrypted communication.

For another example, the method can include storing the dosing parameters in a memory of the drug administration device. In at least some embodiments, storing the dosing parameters in a memory includes updating dosing parameters stored in the memory.

For yet another example, the relevant subset of dosing parameter data indicates which set of a plurality of sets of dosing parameters to adopt.

For another example, the drug can include at least one of infliximab, golimumab, ustekinumab, daratumumab, guselkumab, epoetin alfa, risperidone, esketamine, ketamine, and paliperidone palmitate.

For yet another example, the method can include a method of operating a drug administration device that includes establishing dosing parameters for the drug administration device described, assessing a suitability of the dosing parameters, and, if the dosing parameters are not suitable, preventing dispensing of a drug, and, if the dosing parameters are suitable, dispensing a drug based on the dosing parameters. In at least some embodiments, assessing the suitability of the dosing parameters can include comparing the dosing parameters with acceptable dosing parameters stored in a memory of the drug administration device.

For another example, the method can include assessing authenticity of the dosing parameter data by comparing authentication data associated with the dosing parameter data with acceptable dosing parameter authentication data. For yet another example, the method can include assessing authenticity of the drug holder data by comparing authentication data associated with the drug holder data with acceptable drug holder authentication data. For another example, the dosing parameters can include at least one of a volume of drug to be dispensed, a frequency of drug dispensing, a rate of drug dispensing, and a time for the drug to be dispensed. For yet another example, the drug holder data can include an indication of at least one of a type of drug in the drug holder, a concentration of drug in the drug holder, a provenance of drug in the drug holder, and a volume of drug in the drug holder. For another example, the method can include assessing authenticity of the drug holder by comparing authentication data associated with the drug holder with acceptable drug holder authentication data. For still another example, the method can include assessing authenticity of the drug administration device by comparing authentication data associated with the drug administration device with acceptable drug administration device authentication data.

In another aspect, a drug administration device is provided that in one embodiment includes a drug holder configured to hold a drug and including a data storage component configured to store drug holder data. The drug administration device also includes a first communications interface configured to receive the drug holder data from the data storage component, a processor configured to receive the drug holder data from the first communications interface, a second communications interface configured to receive at least a relevant subset of dosing parameter data from a server, a memory configured to store dosing parameters, and a dispensing mechanism configured to dispense the drug from the drug holder based on the dosing parameters established by the relevant subset of dosing.

The drug administration device can vary in any number of ways. For example, the processor can be configured to determine a relevant subset of the dosing parameter data based on the drug holder data. For still another example, the memory can be configured to store a plurality of sets of dosing parameters, and the relevant subset of the dosing parameter data can indicate which set of the plurality of sets of dosing parameters is adopted for operating the dispensing mechanism. For another example, the dosing parameters can include at least one of a volume of drug to be dispensed, a frequency of drug dispensing, a rate of drug dispensing, and a time for the drug to be dispensed. For yet another example, the drug holder data can include an indication of at least one of a type of drug in the drug holder, a concentration of drug in the drug holder, a provenance of drug in the drug holder, and a volume of drug in the drug holder. For still another example, the memory can be configured to store authentication data. For another example, the drug can include at least one of infliximab, golimumab, ustekinumab, daratumumab, guselkumab, epoetin alfa, risperidone, esketamine, ketamine, and pabperidone palmitate.

In another aspect, a drug administration system is provided that in one embodiment includes a drug administration device that includes a drug holder configured to hold the drug and that includes a dispensing mechanism configured to dispense the drug from the drug holder. The system further includes an external device configured to acquire drug holder data associated with the drug holder. The external device includes a first communications interface configured to receive at least a relevant subset of dosing parameter data from a server, a processor configured to receive the drug holder data, and a memory configured to store dosing parameters. The dispensing mechanism is configured to dispense the drug from the drug holder based on dosing parameters established by the relevant subset of dosing parameter data.

The drug administration system can have any number of variations. For example, the drug holder can include a data storage component configured to store the drug holder data, and the external device can include a second communications interface configured to receive the drug holder data. For another example, the external device can include an image sensor configured to acquire the drug holder data. For yet another example, the processor can be configured to determine a relevant subset of the dosing parameter data based on the drug holder data. For still another example, the memory can be configured to store a plurality of sets of dosing parameters, and the relevant subset of the dosing parameter data can indicate which set of the plurality of sets of dosing parameters is adopted for operating the dispensing mechanism. For another example, the dosing parameters can include at least one of a volume of drug to be dispensed, a frequency of drug dispensing, a rate of drug dispensing, and a time for the drug to be dispensed. For yet another example, the drug holder data can include an indication of at least one of a type of drug in the drug holder, a concentration of drug in the drug holder, a provenance of drug in the drug holder, and a volume of drug in the drug holder. For still another example, the memory can be configured to store authentication data. For another example, the drug can include at least one of infliximab, golimumab, ustekinumab, daratumumab, guselkumab, epoetin alfa, risperidone, esketamine, ketamine, and pabperidone palmitate.

In another embodiment, a drug administration system includes a drug administration device that includes a housing, a drug holder configured to hold a drug and being at least partially within the housing, and a dispensing mechanism configured to dispense a drug and being at least partially within the housing. The system also includes an external device configured to acquire first component data relating to a first component of the drug administration device and including a processor configured to receive and compare the first component data with acceptable first component data and set an operational status of the drug administration device based on the comparison.

The system can have any number of variations. For example, the external device can also include an image sensor configured to image the drug administration device, and the first component data can include image data. For another example, the drug administration device can also include data storage configured to contain the first component data.

For yet another example, the data storage can be attached to the drug holder. For still another example, the first component data can include an indication of at least one of a type of drug in the drug holder, a provenance of the drug, a concentration of drug in the drug holder, an expiry date of the drug, and a volume of drug in the drug holder.

For another example, the data storage can be associated with the housing or the drug dispensing mechanism. In at least some embodiments, the first component data can include an indication of capabilities of the drug dispensing mechanism. The first component data can include an indication of compatible drug holder parameters.

For another example, the drug holder can be removable from the drug administration device. For yet another example, the drug can include at least one of infliximab, golimumab, ustekinumab, daratumumab, guselkumab, epoetin alfa, risperidone, esketamine, ketamine, and paliperidone palmitate.

In another aspect, a method of establishing compatibility of components of a drug administration device is provided that in one embodiment includes acquiring first component data relating to a first component of the drug administration device, comparing the first component data with acceptable first component data, and setting an operational status of the drug administration device based on the comparison of first component data with acceptable first component data.

The method can vary in any number of ways. For example, acquiring the first component data can include communicating the first component data from the first component to an external device. In at least some embodiments, comparing the first component data with acceptable first component data can occurs on external device. The first component data can include image data, and acquiring the first component data can include imaging the drug administration device an external device.

For another example, acquiring the first component data can be carried out on an external device. In at least some embodiments, comparing the first component data with acceptable first component data can occurs on external device. The first component data can include image data, and acquiring the first component data can include imaging the drug administration device an external device.

For yet another example, comparing the first component data with acceptable first component data can utilize a processor of the drug administration device. In at least some embodiments, the first component can include the processor. Comparing the first component data with acceptable first component data can utilize the processor.

For still another example, acquiring the first component data can include communicating the first component data from the first component to a processor of the drug administration device. In at least some embodiments, the first component can include the processor. Comparing the first component data with acceptable first component data can utilize the processor.

For another example, setting the operational status of the drug administration device can include either flagging that the operational status should be fully operational when the first component data corresponds with acceptable first component data and flagging that the operational status should not be fully operational when the first component data does not correspond with acceptable first component data, or setting the operational status as fully operational when all required flags indicate that the operational status should be fully operational and setting the operational status as not fully operational when any required flags indicate the operational status should not be fully operational.

For yet another example, the first component can be a drug holder configured to hold a drug. In at least some embodiments, the first component data can include an indication of at least one of a type of drug in the drug holder, a provenance of the drug, a concentration of drug in the drug holder, an expiry date of the drug, and a volume of drug in the drug holder. The first component data can include an indication of compatible drug dispensing mechanism parameters.

For another example, the first component can include a drug dispensing mechanism. In at least some embodiments, the first component data can include an indication of capabilities of the drug dispensing mechanism. The first component data can include an indication of compatible drug holder parameters.

For still another example, the first component data can be encoded on the first component.

For yet another example, the drug can include at least one of infliximab, golimumab, ustekinumab, daratumumab, guselkumab, epoetin alfa, risperidone, esketamine, ketamine, and paliperidone palmitate.

In another aspect, a drug administration device is provided that in one embodiment includes a housing, a drug holder configured to hold the drug and being at least partially within the housing, a dispensing mechanism configured to dispense the drug and being at least partially within the housing, data storage configured to contain first component data, and a processor configured to receive and compare the first component data with acceptable first component data and set an operational status of the drug administration device based on the comparison.

The drug administration device can have any number of variations. For example, the data storage can be attached to the drug holder. For another example, the first component data can include an indication of at least one of a type of drug in the drug holder, a provenance of the drug, a concentration of drug in the drug holder, an expiry date of the drug, and a volume of drug in the drug holder.

For yet another example, the data storage can be associated with the housing or the drug dispensing mechanism. In at least some embodiments, the first component data can include an indication of capabilities of the drug dispensing mechanism. The first component data can include an indication of compatible drug holder parameters.

For another example, the drug holder can be removable from the drug administration device. For yet another example, the drug can include at least one of infliximab, golimumab, ustekinumab, daratumumab, guselkumab, epoetin alfa, risperidone, esketamine, ketamine, and paliperidone palmitate.

### BRIEF DESCRIPTION OF DRAWINGS

The present invention is described by way of reference to the accompanying figures which are as follows:

FIG. 1 is a schematic view of a first type of drug administration device, namely an auto injector;

FIG. 2 is a schematic view of a second type of drug administration device, namely an infusion pump;

FIG. 3 is a schematic view of a third type of drug administration device, namely an inhaler;

FIG. 4 is a schematic view of a fourth type of drug administration device, namely a nasal spray device;

FIG. 5A is a schematic view of a general drug administration device;

FIG. 5B is a schematic view of a universal drug administration device;

FIG. 6 is a schematic view of a housing for a dosage form;

FIG. 7 is a schematic view of one embodiment of a communication network system with which the drug administration devices and housing can operate;

FIG. 8 is a schematic view of one embodiment of a computer system with which the drug administration devices and housing can operate;

FIG. 9 depicts one embodiment of a method of establishing dosing parameters;

FIG. 10 schematically depicts one embodiment of transfer of data between various elements;

FIG. 11 is a schematic view of one embodiment of a drug administration device showing a cutaway view to reveal a drug holder within a housing;

FIG. 12 is a schematic view of one embodiment of an external device for acquiring image data of components of a drug administration device;

FIG. 13 depicts one embodiment of a method of establishing compatibility of components;

FIG. 14 is a schematic view of another embodiment of a drug administration device; and

FIG. 15 is a schematic view of part of one embodiment of a drug administration system.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Examples of various types of drug administration devices, namely: an autoinjector 100, an infusion pump 200, an inhaler 300, and a nasal spray device 400, are described below with reference to the hereinbefore referenced figures.

### Autoinjector

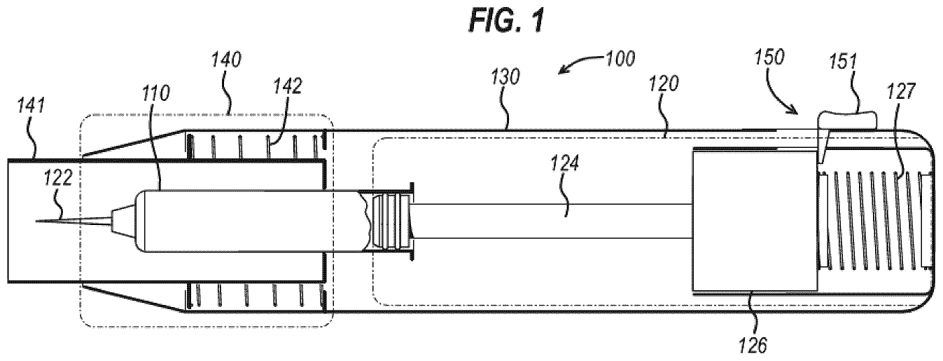

FIG. 1 is a schematic exemplary view of a first type of drug delivery device, namely an injection device, in this example an autoinjector 100, useable with embodiments described herein. The autoinjector 100 comprises a drug holder 110 which retains a drug to be dispensed and a dispensing mechanism 120 which is configured to dispense a drug from the drug holder 110 so that it can be administered to a patient. The drug holder 110 is typically in the form of a container which contains the drug, for example it may be provided in the form of a syringe or a vial, or be any other suitable container which can hold the drug. The autoinjector 100 comprises a discharge nozzle 122, for example a needle of a syringe, which is provided at a distal end of the drug holder 110. The dispensing mechanism 120 comprises a drive element 124, which itself may also comprise a piston and/or a piston rod, and drive mechanism 126. The dispensing mechanism 120 is located proximal to the end of the drug holder 110 and towards the proximal end of the autoinjector 100.

The autoinjector 100 comprises a housing 130 which contains the drug holder 110, drive element 124 and drive mechanism 126 within the body of the housing 130, as well as containing the discharge nozzle 122, which, prior to injection, would typically be contained fully within the housing, but which would extend out of the housing 130 during an injection sequence to deliver the drug. The dispensing mechanism 120 is arranged so that the drive element 124 is advanced through the drug holder 110 in order to dispense the drug through the discharge nozzle 122, thereby allowing the autoinjector to administer a drug retained in drug holder 110 to a patient. In some instances, a user may advance the drive element 124 through the drug holder 110 manually. In other instances, the drive mechanism 126 may include a stored energy source 127 which advances the drive element 124 without user assistance. The stored energy source 127 may include a resilient biasing member such as a spring, or a pressurized gas, or electronically powered motor and/or gearbox.

The autoinjector 100 includes a dispensing mechanism protection mechanism 140. The dispensing mechanism protection mechanism 140 typically has two functions. Firstly, the dispensing mechanism protection mechanism 140 can function to prevent access to the discharge nozzle 122 prior to and after injection. Secondly, the autoinjector 100 can function, such that when put into an activated state, e.g., the dispensing mechanism protection mechanism 140 is moved to an unlocked position, the dispensing mechanism 120 can be activated.

The protection mechanism 140 covers at least a part of the discharge nozzle 122 when the drug holder 110 is in its retracted position proximally within the housing 130. This is to impede contact between the discharge nozzle 122 and a user. Alternatively, or in addition, the protection mechanism 140 is itself configured to retract proximally to expose the discharge nozzle 122 so that it can be brought into contact with a patient. The protection mechanism 140 comprises a shield member 141 and return spring 142. Return spring 142 acts to extend the shield member 141 from the housing 130, thereby covering the discharge nozzle 122 when no force is applied to the distal end of the protection mechanism 140. If a user applies a force to the shield member 141 against the action of the return spring 142 to overcome the bias of the return spring 142, the shield member 141 retracts within the housing 130, thereby exposing the discharge nozzle 122. The protection mechanism 140 may alternatively, or in addition, comprise an extension mechanism (not shown) for extending the discharge nozzle 122 beyond the housing 130, and may further comprise a retracting mechanism (not shown) for retracting the discharge nozzle 122 within the housing 130. The protection mechanism 140 may alternatively, or in addition, comprise a housing cap and/or discharge nozzle boot, which can be attached to the autoinjector 100. Removal of the housing cap would typically also remove the discharge nozzle boot from the discharge nozzle 122.

The autoinjector 100 also includes a trigger 150. The trigger 150 comprises a trigger button 151 which is located on an external surface of the housing 130 so that it is accessible by a user of the autoinjector 100. When the trigger 150 is pressed by a user, it acts to release the drive mechanism 126 so that, via the drive element 124, the drug is then driven out of the drug holder 110 via the discharge nozzle 122.

The trigger 150 may also cooperate with the shield member 141 in such a way that the trigger 150 is prevented from being activated until the shield member 141 has been retracted proximally sufficiently into the housing 130 into an unlocked position, for example by pushing a distal end of the shield member 141 against the skin of a patient. When this has been done, the trigger 150 becomes unlocked, and the autoinjector 100 is activated such that the trigger 150 can be depressed and the injection and/or drug delivery sequence is then initiated. Alternatively, retraction of the shield member 141 alone in a proximal direction into the housing 130 can act to activate the drive mechanism 126 and initiate the injection and/or drug delivery sequence. In this way, the autoinjector 100 has device operation prevention mechanism which prevents dispensing of the drug by, for example, preventing accidental release of the dispensing mechanism 120 and/or accidental actuation of the trigger 150.

While the foregoing description relates to one example of an autoinjector, this example is presented purely for illustration, the present invention is not limited solely to such an autoinjector. A person skilled in the art understands that various modifications to the described autoinjector may be implemented within the scope of the present disclosure.

Auto injectors of the present disclosure can be used to administer any of a variety of drugs, such as any of epinephrine, Rebif, Enbrel, Aranesp, atropine, pralidoxime chloride, and diazepam.

### Infusion Pump

In other circumstances, patients can require precise, continuous delivery of medication or medication delivery on a regular or frequent basis at set periodic intervals. Infusion pumps can provide such controlled drug infusion, by facilitating the administering of the drug at a precise rate that keeps the drug concentration within a therapeutic margin, without requiring frequent attention by a healthcare professional or the patient. In this manner the infusion pump can operate automatically without the manual input of the user for administering the drug.

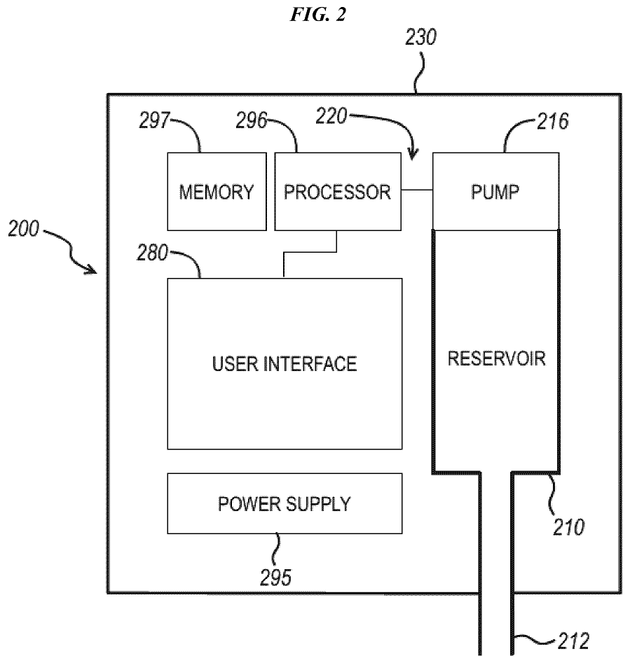

FIG. 2 is a schematic exemplary view of a second type of drug delivery device, namely an infusion pump 200, useable with the embodiments described herein. The infusion pump 200 comprises a drug holder 210 in the form of a reservoir for containing a drug to be delivered, and a dispensing mechanism 220 comprising a pump 216 adapted to dispense a drug contained in the reservoir, so that the drug can be delivered to a patient. These components of the infusion pump are located within housing 230. The dispensing mechanism 220 further comprises an infusion line 212. The drug is delivered from the reservoir upon actuation of the pump 216 via the infusion line 212, which may take the form of a cannula. The pump 216 may take the form of an elastomeric pump, a peristaltic pump, an osmotic pump, or a motor-controlled piston in a syringe. Typically, the drug is delivered intravenously, although subcutaneous, arterial and epidural infusions may also be used.

Infusion pumps of the present disclosure can be used to administer any of a variety of drugs, such as any of insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, and zoledronic acid.

The infusion pump 200 further comprises control circuitry, for example a processor 296 in addition to a memory 297 and a user interface 280, which together provide a triggering mechanism and/or dosage selector for the pump 200. The user interface 280 may be implemented by a display screen located on the housing 230 of the infusion pump 200. The control circuitry and user interface 280 can be located within the housing 230, or external thereto and communicate via a wired or wireless interface with the pump 216 to control its operation.

Actuation of the pump 216 is controlled by the processor 296 which is in communication with the pump 216 for controlling the pump's operation. The processor 296 may be programmed by a user (e.g., patient or healthcare professional), via a user interface 280. This enables the infusion pump 200 to deliver the drug to a patient in a controlled manner. The user can enter parameters, such as infusion duration and delivery rate. The delivery rate may be set by the user to a constant infusion rate or as set intervals for periodic delivery, typically within pre programmed limits. The programmed parameters for controlling the pump 216 are stored in and retrieved from the memory 297 which is in communication with the processor 296. The user interface 280 may take the form of a touch screen or a keypad. In line with the present invention, the established dosing parameters may be used by the processor to control delivery of the drug.

A power supply 295 provides power to the pump 216, and may take the form of an energy source which is integral to the pump 216 and/or a mechanism for connecting the pump 216 to an external source of power.

The infusion pump 200 may take on a variety of different physical forms depending on its designated use. It may be a stationary, non-portable device, e.g., for use at a patient's bedside, or it may be an ambulatory infusion pump which is designed to be portable or wearable. An integral power supply 295 is particularly beneficial for ambulatory infusion pumps.

Whilst the foregoing description relates to one example of an infusion pump, this example is provided purely for illustration. The present disclosure is not limited to such an infusion pump. A person skilled in the art understands that various modifications to the described infusion pump may be implemented within the scope of the present disclosure. For example, the processor may be pre-programmed, such that it is not necessary for the infusion pump to include a user interface.

### Inhaler

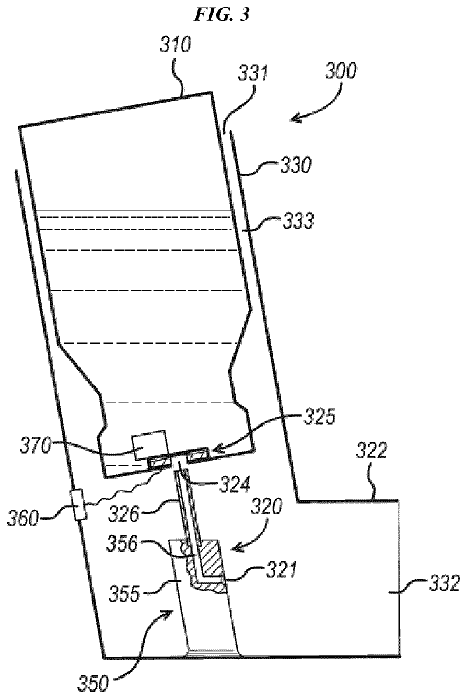

FIG. 3 is a schematic view of a third type of drug administration device, namely an inhaler 300. Inhaler 300 includes a drug holder 310 in the form of a canister. The drug holder 310 contains a drug that would typically be in solution or suspension with a suitable carrier liquid. The inhaler 300 further comprises a dispensing mechanism 320, which includes a pressurized gas for pressurizing the drug holder 310, a valve 325 and nozzle 321. The valve 325 forms an outlet of the drug holder 310. The valve 325 comprises a narrow opening 324 formed in the drug holder 310 and a movable element 326 that controls the opening 324. When the movable element 326 is in a resting position, the valve 325 is in a closed or unactuated state in which the opening 324 is closed and the drug holder 310 is sealed. When the movable element 326 is actuated from the resting position to an actuated position, the valve 325 is actuated into an open state in which the opening 324 is open. Actuation of the movable element 326 from the resting position to the actuated position comprises moving the movable element 326 into the drug holder 310. The movable element 326 is resiliently biased into the resting position. In the open state of the valve 325, the pressurized gas propels the drug in solution or suspension with the suitable liquid out of the drug holder 310 through the opening 324 at high speed. The high speed passage of the liquid through the narrow opening 324 causes the liquid to be atomized, that is, to transform from a bulk liquid into a mist of fine droplets of liquid and/or into a gas cloud. A patient may inhale the mist of fine droplets and/or the gas cloud into a respiratory passage. Hence, the inhaler 300 is capable of delivering a drug retained within the drug holder 310 into a respiratory passage of a patient.

The drug holder 310 is removably held within a housing 330 of the inhaler 300. A passage 333 formed in the housing 330 connects a first opening 331 in the housing 330 and a second opening 332 in the housing 330. The drug holder 310 is received within the passage 333. The drug holder 310 is slidably insertable through the first opening 331 of the housing 330 into the passage 333. The second opening 332 of the housing 330 forms a mouthpiece 322 configured to be placed in a patient's mouth or a nosepiece configured to be placed in a patient's nostril, or a mask configured to be placed over the patient's mouth and nose. The drug holder 310, the first opening 331 and the passage 333 are sized such that air can flow through the passage 333, around the drug holder 310, between the first opening 331 and the second opening 332. The inhaler 300 may be provided with a dispensing mechanism protection mechanism 140 in the form of a cap (not shown) which can be fitted to the mouthpiece 322.

Inhaler 300 further comprises a trigger 350 including a valve actuation feature 355 configured to actuate the valve 325 when the trigger 350 is activated. The valve actuation feature 355 is a projection of the housing 330 into the passage 333. The drug holder 310 is slidably movable within the passage 333 from a first position into a second position. In the first position, an end of the movable element 326 in the resting position abuts the valve actuation feature 355. In the second position, the drug holder 310 can be displaced towards the valve actuation feature 355 such that the valve actuation feature 355 moves the movable element 326 into the drug holder 310 to actuate the valve 325 into the open state. The user's hand provides the necessary force to move the drug holder 310 from the first position to the second position against the resiliently biased movable element 326. The valve actuation feature 355 includes an inlet 356, which is connected to the nozzle 321. The inlet 356 of the valve actuation feature 355 is sized and positioned to couple to the opening 324 of the valve 325 such that the ejected mist of droplets and/or gas cloud can enter the inlet 356 and exit from the nozzle 321 into the passage 333. The nozzle 321 assists in the atomization of the bulk liquid into the mist of droplets and/or gas cloud.

The valve 325 provides a metering mechanism 370. The metering mechanism 370 is configured to close the valve after a measured amount of liquid, and therefore, drug, has passed through the opening 324. This allows a controlled dose to be administered to the patient. Typically, the measured amount of liquid is pre-set, however, the inhaler 300 may be equipped with a dosage selector 360 that is user operable to change the defined amount of liquid. The inhaler may comprise a device indicator in order to alert the user to the required dose. The user can then set the dose accordingly.

While the foregoing description relates to one particular example of an inhaler, this example is purely illustrative. The description should not be seen as limited only to such an inhaler. A person skilled in the art understands that numerous other types of inhaler and nebulizers may be used with the present disclosure. For example, the drug may be in a powdered form, the drug may be in liquid form, or the drug may be atomized by other forms of dispensing mechanism 320 including ultrasonic vibration, compressed gas, a vibrating mesh, or a heat source.

The inhalers of the present disclosure can be used to administer any of a variety of drugs, such as any of mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

### Nasal Spray Device

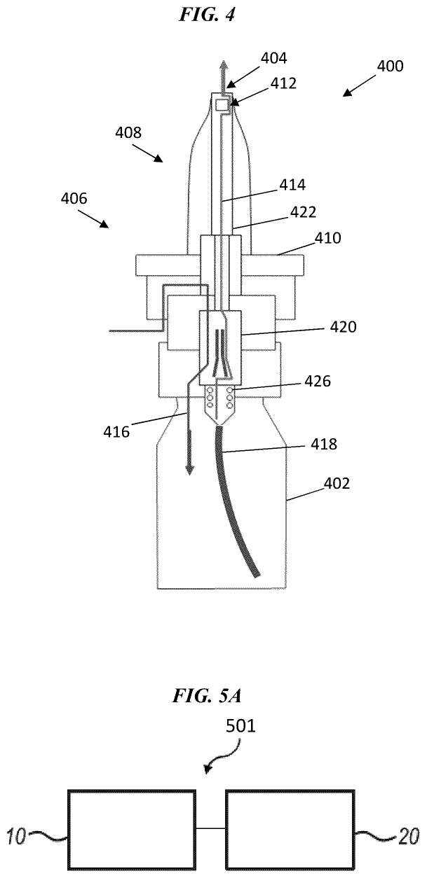

FIG. 4 is a schematic view of a fourth type of drug administration device, namely a nasal spray device 400. The nasal spray device 400 is configured to expel a drug into a nose of a patient. The nasal spray device 400 includes a drug holder 402 configured to contain a drug therein for delivery from the device 400 to a patient. The drug holder 102 can have a variety of configurations, such as a bottle reservoir, a cartridge, a vial (as in this illustrated embodiment), a blow-fill-seal (BFS) capsule, a blister pack, etc. In an exemplary embodiment, the drug holder 402 is a vial. An exemplary vial is formed of one or more materials, e.g., glass, polymer(s), etc. In some embodiments, a vial can be formed of glass. In other embodiments, a vial can be formed of one or more polymers. In yet other embodiments, different portions of a vial can be formed of different materials. An exemplary vial can include a variety of features to facilitate sealing and storing a drug therein, as described herein and illustrated in the drawings. However, a person skilled in the art will appreciate that the vials can include only some of these features and/or can include a variety of other features known in the art. The vials described herein are merely intended to represent certain exemplary embodiments.

An opening 404 of the nasal spray device 400 through which the drug exits the nasal spray device 400 is formed in in a dispensing head 406 of the nasal spray device 400 in a tip 408 of the dispensing head 406. The tip 408 is configured to be inserted into a nostril of a patient. In an exemplary embodiment, the tip 408 is configured to be inserted into a first nostril of the patient during a first stage of operation of the nasal spray device 400 and into a second nostril of the patient during a second stage of operation of the nasal spray device 400. The first and second stages of operation involve two separate actuations of the nasal spray device 400, a first actuation corresponding to a first dose of the drug being delivered and a second actuation corresponding to a second dose of the drug being delivered. In some embodiments, the nasal spray device 400 is configured to be actuated only once to deliver one nasal spray. In some embodiments, the nasal spray device 400 is configured to be actuated three or more times to deliver three or more nasal sprays, e.g., four, five, six, seven, eight, nine, ten, etc.

The dispensing head 406 includes a depth guide 410 configured to contact skin of the patient between the patient's first and second nostrils, such that a longitudinal axis of the dispensing head 406 is substantially aligned with a longitudinal axis of the nostril in which the tip 408 is inserted. A person skilled in the art will appreciate that the longitudinal axes may not be precisely aligned but nevertheless be considered to be substantially aligned due to any number of factors, such as manufacturing tolerances and sensitivity of measurement equipment.

In an exemplary embodiment, as in FIG. 4, the dispensing head 406 has a tapered shape in which the dispensing head 406 has a smaller diameter at its distal end than at its proximal end where the opening 404 is located. The opening 404 having a relatively small diameter facilitates spray of the drug out of the opening 404, as will be appreciated by a person skilled in the art. A spray chamber 412 through which the drug is configured to pass before exiting the opening 404 is located within a proximal portion of the tapered dispensing head 406, distal to the opening 404. When the drug passes through the spray chamber 412 at speed, the spray chamber 412 facilitates production of a fine mist that exits through the opening 404 with a consistent spray pattern. Arrow 414 in FIG. 4 illustrates a path of travel of the drug from the drug holder 402 and out of the opening 404.

In some embodiments, the dispensing head 406 can include two tips 408 each having an opening 404 therein such that the nasal spray device 400 is configured to simultaneously deliver doses of drug into two nostrils in response to a single actuation.

The dispensing head 406 is configured to be pushed toward the drug holder 402, e.g., depressed by a user pushing down on the depth guide 410, to actuate the nasal spray device 400. In other words, the dispensing head 406 is configured as an actuator to be actuated to drive the drug from the drug holder 402 and out of the nasal spray device 400. In an exemplary embodiment, the nasal spray device 400 is configured to be self-administered such that the user who actuates the nasal spray device 400 is the patient receiving the drug from the nasal spray device 400, although another person can actuate the nasal spray device 400 for delivery into another person.

The actuation, e.g., depressing, of the dispensing head 406 is configured to cause venting air to enter the drug holder 402, as shown by arrow 416 in FIG. 4. The air entering the drug holder 402 displaces drug in the drug holder through a tube 418 and then into a metering chamber 420, which displaces drug proximally through a cannula 422, through the spray chamber 412, and then out of the opening 404. In response to release of the dispensing head 406, e.g., a user stops pushing downward on the dispensing head 406, a bias spring 426 causes the dispensing head 406 to return to its default, resting position to position the dispensing head 406 relative to the drug holder 402 for a subsequent actuation and drug delivery.

While the foregoing description relates to one particular example of a nasal spray device, this example is purely illustrative. The description should not be seen as limited only to such a nasal spray device. A person skilled in the art understands that the nasal spray device 400 can include different features in different embodiments depending upon various requirements. For example, the nasal spray device 400 can lack the depth guide 410 and/or may include any one or more of a device indicator, a sensor, a communications interface, a processor, a memory, and a power supply.

The nasal spray devices of the present disclosure can be used to administer any of a variety of drugs, such as any of ketamine (e.g., Ketalar®), esketamine (e.g., Spravato®, Ketanest®, and Ketanest-SC)), naloxone (e.g., Narcan®), and sumatriptan (e.g., Imitrex®).

### Drug Administration Device

As will be appreciated from the foregoing, various components of drug delivery devices are common to all such devices. These components form the essential components of a universal drug administration device. A drug administration device delivers a drug to a patient, where the drug is provided in a defined dosage form within the drug administration device.

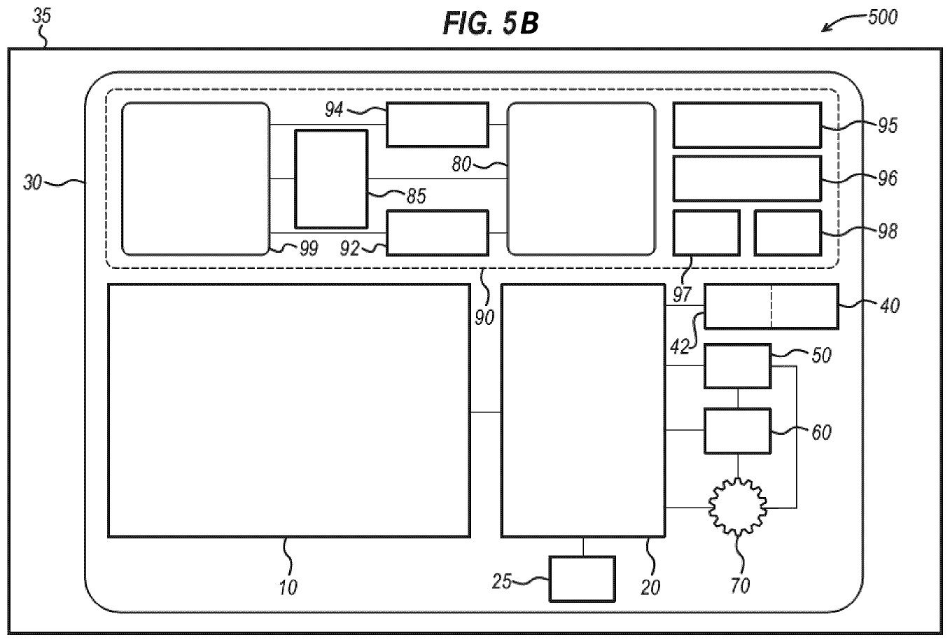

FIG. 5A is a generalized schematic view of such a universal drug administration device 501, and FIG. 5B is an exemplary embodiment of such a universal drug administration device 500. Examples of the universal drug administration device 500 include injection devices (e.g., autoinjectors, jet injectors, and infusion pumps), nasal spray devices, and inhalers.

As shown in FIG. 5A, drug administration device 501 includes in general form the features of a drug holder 10 and a dispensing mechanism 20. The drug holder 10 holds a drug in a dosage form to be administered. The dispensing mechanism 20 is configured to release the dosage form from the drug holder 10 so that the drug can be administered to a patient.

FIG. 5B shows a further universal drug administration device 500 which includes a number of additional features. A person skilled in the art understands that these additional features are optional for different embodiments, and can be utilized in a variety of different combinations such that the additional features may be present or may be omitted from a given embodiment of a particular drug administration device, depending upon requirements, such as the type of drug, dosage form of the drug, medical indication being treated with the drug, safety requirements, whether the device is powered, whether the device is portable, whether the device is used for self-administration, and many other requirements which will be appreciated by a person skilled in the art. Similar to the universal device of FIG. 5A, the drug administration device 500 comprises a housing 30 which accommodates the drug holder 10 and dispensing mechanism 20.

The device 500 is provided with a triggering mechanism 50 for initiating the release of the drug from the drug holder 10 by the dispensing mechanism 20. The device 500 includes the feature of a metering/dosing mechanism 70 which measures out a set dose to be released from the drug holder 10 via the dispensing mechanism 20. In this manner, the drug administration device 500 can provide a known dose of determined size. The device 500 comprises a dosage selector 60 which enables a user to set the dose volume of drug to be measured out by the metering mechanism 50. The dose volume can be set to one specific value of a plurality of predefined discrete dose volumes, or any value of predefined dose volume within a range of dose volumes. The drug administration device may be configured to adjust the dose volume without intervention by the user.

The device 500 can comprise a device operation prevention mechanism 40 or 25 which when in a locked state will prevent and/or stop the dispensing mechanism 20 from releasing the drug out of the drug holder 10, and when in an unlocked state will permit the dispensing mechanism 20 to release the drug dosage from out of the drug holder 10. This can prevent accidental administration of the drug, for example to prevent dosing at an incorrect time, or for preventing inadvertent actuation. The device 500 also includes a dispensing mechanism protection mechanism 42 which prevents access to at least a part of the dispensing mechanism 20, for example for safety reasons. Device operation prevention mechanism 40 and dispensing mechanism protection mechanism 42 may be the same component. Each of the device operation prevention mechanism and the dispensing mechanism protection mechanism may be enabled and disabled by the drug administration device without intervention of the user.

The device 500 can include a device indicator 85 which is configured to present information about the status of the drug administration device and/or the drug contained therein. The device indicator 85 may be a visual indicator, such as a display screen, or an audio indicator. The device 500 includes a user interface 80 which can be configured to present a user of the device 500 with information about the device 500 and/or to enable the user to control the device 500. The device indicator 85 can be used to prompt a user to select the required dosing parameters based on the established dosing parameters. The device indicator 85 can prompt the user to administer the drug when required by the established dosing parameters. The device 500 includes a device sensor 92 which is configured to sense information relating to the drug administration device and/or the drug contained therein, for example dosage form and device parameters. As an example, in embodiments which include a metering mechanism 70 and a dosage selector 60, the embodiment may further include one or more device sensors 92 configured to sense one or more of: the dose selected by a user using dosage selector 60, the dose metered by the metering mechanism 70 and the dose dispensed by the dispensing mechanism 20.

Similarly, an environment sensor 94 may be provided which is configured to sense information relating to the environment in which the device 500 is present, such as the temperature of the environment, the humidity of the environment, location, and time. There may be a dedicated location sensor 98 which is configured to determine the geographical location of the device 500, e.g., via satellite position determination, such as GPS. The device 500 may also include a communications interface 99 which can communicate externally data which has been acquired from the various sensors about the device and/or drug.

If required, the device 500 comprises a power supply 95 for delivering electrical power to one or more electrical components of the device 500. The power supply 95 can be a source of power which is integral to device 500 and/or a mechanism for connecting device 500 to an external source of power. The drug administration device 500 also includes a device computer system 90 including processor 96 and memory 97 powered by the power supply 95 and in communication with each other, and optionally with other electrical and control components of the device 500, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

In some embodiments, the drug administration device 500 is enclosed in packaging 35. The packaging 35 may further include a combination of a processor 96, memory 97, user interface 80, device indicator 85, device sensor 92, location sensor 98 and/or environment sensors 94 as described herein, and these may be located externally on the housing of the device 500.

A person skilled in the art will appreciate that the universal drug administration device 500 comprising the drug holder 10 and dispensing mechanism 20 can be provided with a variety of the optional features described above, in a number of different combinations. Moreover, the drug administration device 500 can include more than one drug holder 10, optionally with more than one dispensing mechanism 20, such that each drug holder has its own associated dispensing mechanism 20.

### Drug Dosage Forms

Conventionally, drug administration devices utilize a liquid dosage form. It will be appreciated, however that other dosage forms are available.

One such common dosage form is a tablet. The tablet may be formed from a combination of the drug and an excipient that are compressed together. Other dosage forms are pastes, creams, powders, ear drops, and eye drops.

Further examples of drug dosage forms include dermal patches, drug eluting stents and intrauterine devices. In these examples, the body of the device comprises the drug and may be configured to allow the release of the drug under certain circumstances. For example, a dermal patch may comprise a polymeric composition containing the drug. The polymeric composition allows the drug to diffuse out of the polymeric composition and into the skin of the patient. Drug eluting stents and intrauterine devices can operate in an analogous manner. In this way, the patches, stents and intrauterine devices may themselves be considered drug holders with an associated dispensing mechanism.

Any of these dosage forms can be configured to have the drug release initiated by certain conditions. This can allow the drug to be released at a desired time or location after the dosage form has been introduced into the patient. In particular, the drug release may be initiated by an external stimulus. Moreover, these dosage forms can be contained prior to administration in a housing, which may be in the form of packaging. This housing may contain some of the optional features described above which are utilized with the universal drug administration device 500.

The drug administered by the drug administration devices of the present disclosure can be any substance that causes a change in an organism's physiology or psychology when consumed. Examples of drugs that the drug administration devices of the present disclosure can administer include 5-alpha-reductase inhibitors, 5-aminosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, aminosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, diarylquinobnes, dibenzazepine anticonvulsants, digestive enzymes, dipeptidyl peptidase 4 inhibitors, diuretics, dopaminergic antiparkinsonism agents, drugs used in alcohol dependence, echinocandins, EGFR inhibitors, estrogen receptor antagonists, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, gly copeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, ketolides, laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, macrolide derivatives, macrolides, magnetic resonance imaging contrast media, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, parathyroid hormone and analogs, PARP inhibitors, PCSK9 inhibitors, penicillinase resistant penicillins, penicillins, peripheral opioid receptor antagonists, peripheral opioid receptor mixed agonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, streptomyces derivatives, succinimide anticonvulsants, sulfonamides, sulfonylureas, synthetic ovulation stimulants, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, ultrasound contrast media, upper respiratory combinations, urea anticonvulsants, urea cycle disorder agents, urinary anti-infectives, urinary antispasmodics, urinary pH modifiers, uterotonic agents, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, or VMAT2 inhibitors. The drug administration devices of the present disclosure may administer a drug selected from epinephrine, Rebif, Enbrel, Aranesp, atropine, pralidoxime chloride, diazepam, insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, zoledronic acid, mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

### Drug Housing

As described above, a dosage form can be provided in a holder that is appropriate for the particular dosage form being utilized. For example, a drug in a liquid dosage form can be held prior to administration within a holder in the form of a vial with a stopper, or a syringe with a plunger. A drug in solid or powder dosage form, e.g., as tablets, may be contained in a housing which is arranged to hold the tablets securely prior to administration.

The housing may comprise one or a plurality of drug holders, where each holder contains a dosage form, e.g., the drug can be in a tablet dosage form and the housing can be in the form of a blister pack, where a tablet is held within each of a plurality of holders. The holders being in the form of recesses in the blister pack.

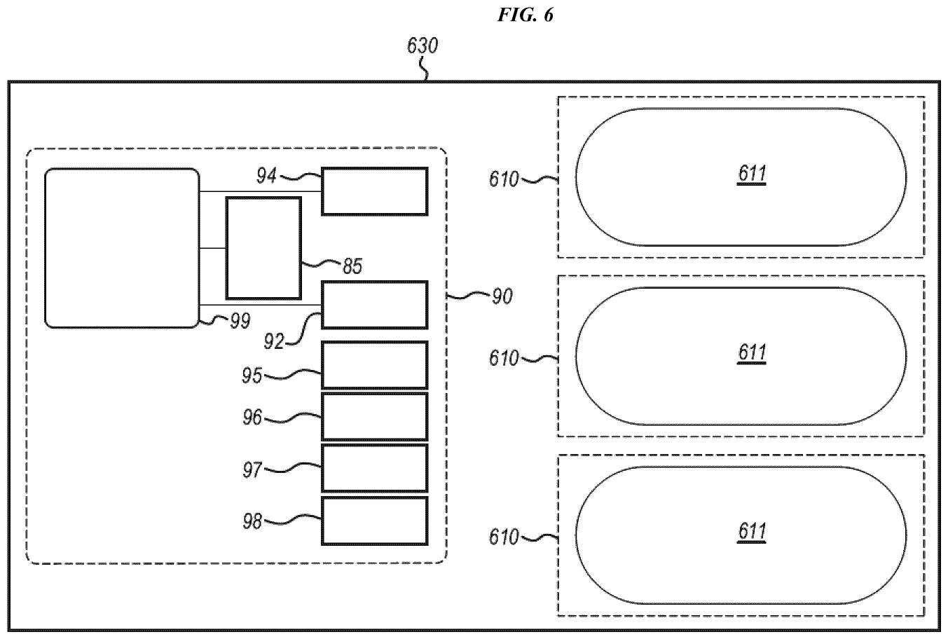

FIG. 6 depicts a housing 630 that comprises a plurality of drug holders 610 that each contain a dosage form 611. The housing 630 may have at least one environment sensor 94, which is configured to sense information relating to the environment in which the housing 630 is present, such as the temperature of the environment, time or location. The housing 630 may include at least one device sensor 92, which is configured to sense information relating to the drug of the dosage form 611 contained within the holder 610. There may be a dedicated location sensor 98 which is configured to determine the geographical location of the housing 630, e.g., via satellite position determination, such as GPS.

The housing 630 may include an indicator 85 which is configured to present information about the status of the drug of the dosage form 611 contained within the holder 610 to a user of the drug housing. The housing 630 may also include a communications interface 99 which can communicate information externally via a wired or wireless transfer of data pertaining to the drug housing 630, environment, time or location and/or the drug itself.

If required, the housing 630 may comprise a power supply 95 for delivering electrical power to one or more electrical components of the housing 630. The power supply 95 can be a source of power which is integral to housing 630 and/or a mechanism for connecting the housing 630 to an external source of power. The housing 630 may also include a device computer system 90 including processor 96 and memory 97 powered by the power supply 95 and in communication with each other, and optionally with other electrical and control components of the housing 630, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

The housing 630 can be in the form of packaging. Alternatively, additional packaging may be present to contain and surround the housing 630.

The holder 610 or the additional packaging may themselves comprise one or more of the device sensor 92, the environment sensor 94, the indicator 85, the communications interface 99, the power supply 95, location sensor 98, and device computer system including the processor 96 and the memory 85, as described above.

### Electronic Communication

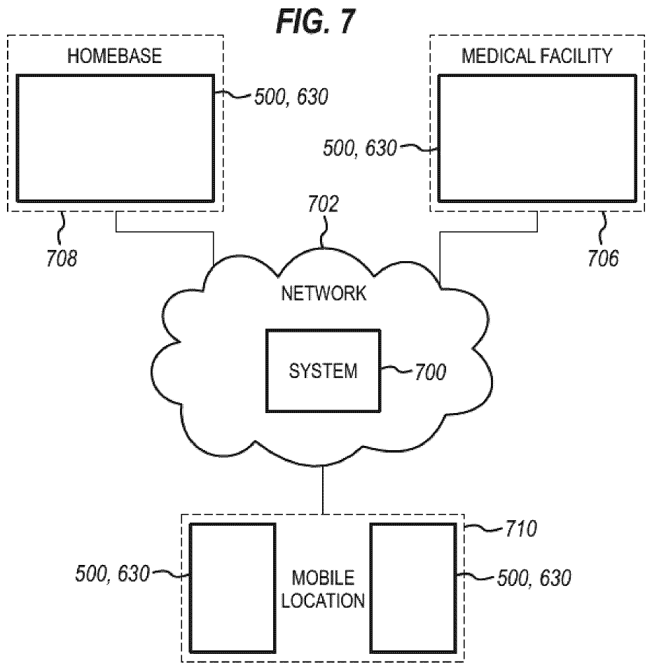

As mentioned above, communications interface 99 may be associated with the drug administration device 500 or drug housing 630, by being included within or on the housing 30, 630, or alternatively within or on the packaging 35. Such a communications interface 99 can be configured to communicate with a remote computer system, such as central computer system 700 shown in FIG. 7. As shown in FIG. 7, the communications interface 99 associated with drug administration device 500 or housing 630 is configured to communicate with a central computer system 700 through a communications network 702 from any number of locations such as a medical facility 706, e.g., a hospital or other medical care center, a home base 708 (e.g., a patient's home or office or a care taker's home or office), or a mobile location 710. The communications interface 99 can be configured to access the system 700 through a wired and/or wireless connection to the network 702. In an exemplary embodiment, the communications interface 99 of FIG. 6 is configured to access the system 700 wirelessly, e.g., through Wi-Fi connection(s), which can facilitate accessibility of the system 700 from almost any location in the world.

A person skilled in the art will appreciate that the system 700 can include security features such that the aspects of the system 700 available to any particular user can be determined based on, e.g., the identity of the user and/or the location from which the user is accessing the system. To that end, each user can have a unique username, password, biometric data, and/or other security credentials to facilitate access to the system 700. The received security parameter information can be checked against a database of authorized users to determine whether the user is authorized and to what extent the user is permitted to interact with the system, view information stored in the system, and so forth.

### Computer System

As discussed herein, one or more aspects or features of the subject matter described herein, for example components of the central computer system 700, processor 96, power supply 95, memory 97, communications interface 99, user interface 80, device indicators 85, device sensors 92, environment sensors 94 and location sensors 98, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computer system may include clients and servers. A client and server are generally remote from each other and typically interact through a communications network, e.g., the Internet, a wireless wide area network, a local area network, a wide area network, or a wired network. The relationship of client and server arises by virtue of computer programs running on the respective computers and having a client-server relationship to each other.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example user interface 80 (which can be integrated or separate to the administration device 500 or housing 630), can be implemented on a computer having a display screen, such as for example a cathode ray tube (CRT) or a liquid crystal display (LCD) or a light emitting diode (LED) monitor for displaying information to the user. The display screen can allow input thereto directly (e.g., as a touch screen) or indirectly (e.g., via an input device such as a keypad or voice recognition hardware and software). Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. As described above, this feedback may be provided via one or more device indicators 85 in addition to the user interface 80. The device indicators 85 can interact with one or more of device sensor(s) 92, environment sensor(s) 94 and/or location sensor(s) 98 in order to provide this feedback, or to receive input from the user.

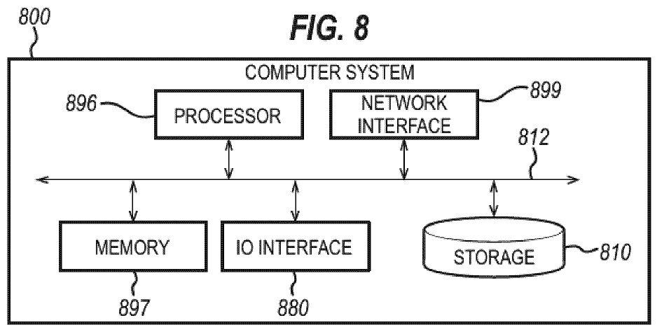

FIG. 8 illustrates one exemplary embodiment of the computer system 700, depicted as computer system 800. The computer system includes one or more processors 896 configured to control the operation of the computer system 800. The processor(s) 896 can include any type of microprocessor or central processing unit (CPU), including programmable general-purpose or special-purpose microprocessors and/or any one of a variety of proprietary or commercially available single or multi-processor systems. The computer system 800 also includes one or more memories 897 configured to provide temporary storage for code to be executed by the processor(s) 896 or for data acquired from one or more users, storage devices, and/or databases. The memory 897 can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The various elements of the computer system are coupled to a bus system 812. The illustrated bus system 812 is an abstraction that represents any one or more separate physical busses, communication lines/interfaces, and/or multi-drop or point-to-point connections, connected by appropriate bridges, adapters, and/or controllers. The computer system 800 also includes one or more network interface(s) 899 (also referred to herein as a communications interface), one or more input/output (TO) interface(s) 880, and one or more storage device(s) 810.

The communications interface(s) 899 are configured to enable the computer system to communicate with remote devices, e.g., other computer systems and/or devices 500 or housings 630, over a network, and can be, for example, remote desktop connection interfaces, Ethernet adapters, and/or other local area network (LAN) adapters. The IO interface(s) 880 include one or more interface components to connect the computer system 800 with other electronic equipment. For example, the IO interface(s) 880 can include high speed data ports, such as universal serial bus (USB) ports, 1394 ports, Wi-Fi, Bluetooth, etc. Additionally, the computer system can be accessible to a human user, and thus the IO interface(s) 880 can include displays, speakers, keyboards, pointing devices, and/or various other video, audio, or alphanumeric interfaces. The storage device(s) 810 include any conventional medium for storing data in a non volatile and/or non-transient manner. The storage device(s) 810 are thus configured to hold data and/or instructions in a persistent state in which the value(s) are retained despite interruption of power to the computer system. The storage device(s) 810 can include one or more hard disk drives, flash drives, USB drives, optical drives, various media cards, diskettes, compact discs, and/or any combination thereof and can be directly connected to the computer system or remotely connected thereto, such as over a network. In an exemplary embodiment, the storage device(s) 810 include a tangible or non-transitory computer readable medium configured to store data, e.g., a hard disk drive, a flash drive, a USB drive, an optical drive, a media card, a diskette, or a compact disc.

The elements illustrated in FIG. 8 can be some or all of the elements of a single physical machine. In addition, not all of the illustrated elements need to be located on or in the same physical machine.

The computer system 800 can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing HyperText Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system 800 can also include a web server for generating and/or delivering the web pages to client computer systems.

As shown in FIG. 7, the computer system 800 of FIG. 8 as described above may form the components of the central computer system 700 which is in communication with one or more of the device computer systems 90 of the one or more individual drug administration devices 500 or housings 630. Data, such as operational data of the devices 500 or housings 630, medical data acquired of patients by such devices 500 or housings 630 can be exchanged between the central and device computer systems 700, 90.

As mentioned the computer system 800 as described above may also form the components of a device computer system 90 which is integrated into or in close proximity to the drug administration device 500 or housing 630. In this regard, the one or more processors 896 correspond to the processor 96, the network interface 899 corresponds to the communications interface 99, the IO interface 880 corresponds to the user interface 80, and the memory 897 corresponds to the memory 97. Moreover, the additional storage 810 may also be present in device computer system 90.

In an exemplary embodiment, the computer system 800 can form the device computer system 90 as a single unit, e.g., contained within a single drug administration device housing 30, contained within a single package 35 for one or more drug administration devices 500, or a housing 630 that comprises a plurality of drug holders 610. The computer system 800 can form the central computer system 700 as a single unit, as a single server, or as a single tower.

The single unit can be modular such that various aspects thereof can be swapped in and out as needed for, e.g., upgrade, replacement, maintenance, etc., without interrupting functionality of any other aspects of the system. The single unit can thus also be scalable with the ability to be added to as additional modules and/or additional functionality of existing modules are desired and/or improved upon.

The computer system can also include any of a variety of other software and/or hardware components, including by way of example, operating systems and database management systems. Although an exemplary computer system is depicted and described herein, it will be appreciated that this is for sake of generality and convenience. In other embodiments, the computer system may differ in architecture and operation from that shown and described here. For non-limiting examples, the memory 897 and storage device 810 can be integrated together or the communications interface 899 can be omitted if communication with another computer system is not necessary.

### Implementations

It can be desirable to monitor compliance with the guidance that is associated with drugs that are administered to a patient in various dosage forms. This can provide assurance that correct procedures are being followed and avoid the adoption of incorrect and potentially dangerous approaches. Further, this may also enable optimization of the administration of the drug to the patient.

Further, it can be desirable to ensure that a correct dosage regimen is used for a particular drug that is present in a drug administration device for delivery to a patient. Ensuring that the drug administration device is in an appropriate state for carrying out the desired administration to the patient may help ensure that the correct dosage regimen is used.

### Establishing Dosing Parameters

In an exemplary embodiment, a method for establishing dosing parameters for a drug administration device includes acquiring drug holder data from a drug holder of the drug administration device and communicating the drug holder data to a processor. Either the drug administration device includes the processor, or an external device includes the processor. The method also includes communicating at least a relevant subset of dosing parameter data from a server to the processor, and establishing the dosing parameters for the drug administration device based on the relevant subset of dosing parameter data. The relevant subset of dosing parameter data is determined based on the drug holder data.

The method for establishing dosing parameters for a drug administration device may ensure that the correct dosing parameters are established for specific details of the drug holder. Further, by obtaining the relevant subset of dosing parameter data from the server it may be possible to ensure that the most up-to-date dosing parameters are established for the drug administration device, which may ensure that an optimal dosing regimen for a patient is utilized regardless of when the drug holder and the drug administration device were issued to the patient.

The drug administration device can be any of the drug administration devices described herein. In particular, the drug administration device can be one that is configured to automatically administer the drug according to the established dosing parameters without intervention from a user of the drug administration device, such as the infusion pump described herein (e.g., infusion pump 200 of FIG. 2).

The drug administration device could be a device that requires a user input in order to initiate drug administration therefrom. The drug administration device can include a device indicator, such as a display screen or an audio indicator as discussed above, that alerts the user to administer the drug. This may allow the drug administration device to prompt the user to enact a dosing regimen based on the established dosing parameters.

The established dosing parameters can relate to any number of parameters associated with administering a dose of the drug from the drug administration device. The established dosing parameters can relate to just one parameter associated with the administration of the drug from the drug administration device, or the established dosing parameters can relate to a plurality of parameters associated with the administration of the drug from the drug administration device.

In an example of a dosing parameter, the dosing parameter can relate to a frequency with which the drug should be administered from the drug administration device. In another example of a dosing parameter, the dosing parameter can indicate particular times at which the drug is to be administered from the drug administration device. In another example of a dosing parameter, the dosing parameter can relate to a volume of drug to be administered by the drug administration device. In another example of a dosing parameter, when applicable to the drug administration device that is utilized, the dosing parameter can refer to a rate at which the drug is administered from the drug administration device. In another example of a dosing parameter, the dosing parameter can relate to a temperature at which the drug can be administered. In this way, the drug may only be administered when the drug is at a safe temperature that is comfortable to the patient, such as after the drug has reached a sufficient temperature after warming up from a cold storage temperature (e.g., when stored in a refrigerator). When the temperature of the drug is one of the relevant dosing parameters, the drug administration device can include an appropriate sensor configured to monitor the temperature of the drug, such as a temperature sensor, e.g., a thermistor, a thermocoupler, a thermistor, etc.

In an exemplary embodiment, the dosing parameters that are established define all of the parameters that can be varied when administering a drug from the drug administration device. In this manner, the established dosing parameters provide a dosage regimen for the drug in the drug holder.

The dosing regimen is an algorithm stored in a memory. The memory can be a memory of the drug administration device, e.g., the memory 97 of the drug administration device 500 of FIG. 5B, that is executable on board the drug administration device by the drug administration device's processor. The memory can be a memory of an external device that is external to, distinct from, and configured to electronically communicate (wired or wirelessly) with the drug administration device.

The algorithm is stored in the form of one or more sets of pluralities of data points defining and/or representing instructions, notifications, signals, etc. to control administration of the drug from the drug administration device. As discussed herein, the drug holder data received by the processor, e.g., as pluralities of data points via a communications interface, can be used by the processor to establish the dosing parameters of the algorithm. The dosing parameters are among the algorithm's data points and are thus each able to be changed by changing one or more of the stored pluralities of data points of the algorithm.

The dosing parameter data received by the processor can identify the dosing parameters to establish and each parameter's updated value. After the dosing parameters have been established, subsequent execution of the algorithm administers another dose of the drug according to the algorithm, which reflects the current dosing regimen.

Establishing the dosing parameters can be automated to improve patient outcomes. Thus, the drug administration device can be configured to facilitate personalized medicine based on the patient to provide a smart system for drug delivery.

The drug administration device can be configured to operate based on the established dosing parameters, e.g., based on the processor's execution of the algorithm that defines the dosing regimen. For example, when the drug administration device is configured to administer the drug without intervention from a user, the established dosing parameters can relate to the time at which the drug dose should be administered. The drug administration device's operation will cause the dose to be administered at the time established by the dosing parameter related to time. As noted herein, the drug administration device can also be configured to adjust the volume of the drug that will be dispensed based on the establishment of a dosing parameter relating to the volume of drug to be administered. Further, the drug administration device can be configured to change its operation based on any other established dosing parameters.

The drug holder of the drug administration device can be of any suitable form for holding the drug. As noted herein, such suitable forms include a vial, a cartridge, or a syringe.

The drug holder includes the drug holder data. The drug holder can include the drug holder data as part of a data storage component that is attached to or otherwise part of the drug holder. For example, the data storage component can be of the form of an integrated circuit configured to communicate the drug holder data from the drug holder. The data storage component can be, for another example, a radio frequency identification (RFID) tag. For yet another example, the data storage component can be in the form of a barcode. The drug holder data can be stored on the drug holder utilizing a single data storage component or a plurality of data storage components. If a plurality of data storage components are used, each can be different from one another, which may help provide redundancy and/or allow for drug holder data retrieval even if a certain type of data communication is currently unavailable, e.g., if an RFID scanner is absent or damaged.

In general, the approach adopted for acquiring the drug holder data depends on the form in which the drug holder data is present. Therefore, acquiring the drug holder data requires the use of an appropriate communications interface for receiving the drug holder data communicated from the drug holder. For example, when the drug holder data is contained as part of an RFID tag, acquiring the drug holder data involves the use of an RFID scanner. For another example, a bar code scanner can be utilized when the drug holder data is contained within the form of a bar code. For yet another example, when the drug holder data is in the form of an integrated circuit, the integrated circuit can be powered and capable of transmitting the drug holder data to a receiving communications interface.

The drug administration device can include the one or more components configured to receive the drug holder data, for example a bar code scanner, an RFID scanner, or other suitable communications interface. This localized setup of sending and receiving the drug holder data may be particularly suitable for drug holder data that is stored in a data storage component where a close proximity between the data storage component and the component for receiving the drug holder data is required for reading the data storage component, such as for a bar code and a bar code reader. Alternatively, the component configured to acquire the drug holder data can be separate to the drug administration device. In particular, the drug holder data can be acquired by the external device. As mentioned above, the external device can include processor that is involved in establishing the dosing parameters, in which case the drug holder data being acquired by the external device may ease the processor's access to the dosing parameters.

The drug holder data being communicated to the processor from the drug holder allows the drug holder data to be part of the computation involved in establishing the dosing parameters carried out by the processor. At least a relevant subset of dosing parameter data is communicated to the processor from the server. In this way, the processor is at least a part of a client in a client-server relationship.

As mentioned above, a relevant subset of the dosing parameter data is communicated to the processor, either only the relevant subset or the entire dosing parameter data that includes the relevant subset as a partial portion thereof, and the processor utilizes the relevant subset of the dosing parameter data in establishing the dosing parameters for the drug administration device. In other words, there may be other dosing parameter data available on the server that is not utilized for establishing the dosing parameters and, in at least some cases, not even transmitted to the processor. The other dosing parameter data may not be used because it is not relevant to the particular drug in the drug holder and/or because it is not relevant to the particular drug administration device. In any case, at least the relevant subset that is utilized for establishing the dosing parameters is communicated from the server to the processor.

In general, an amount of dosing parameter data that is provided from the server to the processor can depend on where the relevant subset of dosing parameter data is determined. If the relevant subset of dosing parameter data is determined on the server, then only the relevant subset of dosing parameter data can be communicated from the server to the processor since the processor would not need any of the other, non-relevant dosing parameter data in establishing the dosing parameters. This avoids the transmission of unnecessary dosing parameter data and so increases efficiency of the communication process. Alternatively, the relevant subset of dosing parameter data along with non-relevant dosing parameter data can be communicated to the processor and the determination of the relevant subset of dosing parameter data be carried out using the processor that is part of the drug administration device or the external device.

The dosing parameter data (whether or not the dosing parameter data includes the non-relevant dosing parameter data) that is communicated from the server to the processor can be stored in a memory at a same location as the processor, e.g., at the drug administration device that includes the processor or at the external device that includes the processor. In this way, any non-relevant dosing parameter data can be stored locally to the processor, so that it may be utilized at a later time. For example, when the drug holder is changed for a different type of drug holder with associated new drug holder data, a previously non-relevant subset of dosing parameter data may now be relevant. By storing dosing parameter data locally, the new dosing parameters can be established without the need to further communicate with the server. Even if non-relevant dosing parameter data is not received and is thus not stored locally, storing the relevant subset of dosing parameter data may facilitate analysis of drug administration device operation for quality control, patient compliance reasons, etc.

As mentioned above, the dosing parameters are established, e.g., using the processor, based on the relevant subset of dosing parameter data, and the relevant subset of dosing parameter data is determined based on the drug holder data. This determination can be performed in a variety of ways. In an exemplary embodiment, the drug holder data identifies each of the parameters of the algorithm that define the dosing regimen and that can be changed by the processor. These changeable parameters of the algorithm are also referred to herein as “variable parameters.” Each of the variable parameters will correspond to one of the dosing parameters in the dosing parameter data. However, one or more of the dosing parameters in the dosing parameter data may not have a corresponding parameter in the dosing regimen algorithm because the parameter is not relevant to the particular drug administration device and/or the particular drug being delivered from the drug administration device. For example, a temperature of the drug may not be relevant because the drug is stored at room temperature and thus does not need time to warm to a temperature for comfortable delivery to a patient. For another example, the drug may be configured to be delivered on demand from the drug administration device such that particular times for the drug to be delivered automatically by the drug administration device is irrelevant. For yet another example, the drug administration device may be configured to deliver only a pre-set dose of the drug such that a volume of drug to be delivered is not relevant. Determining the relevant subset of dosing parameter data can thus include determining which of the dosing parameters in the dosing parameter data has a corresponding parameter in the drug holder data and thus which of the dosing parameters in the dosing parameter data has a corresponding variable parameter in the dosing regimen algorithm. This determination can be a simple comparison, such as by using a lookup table or by comparing identifier data included in the drug holder data that is associated with each parameter to identify which of the dosing parameters in the dosing parameter data has an associated identifier in the dosing parameter data that matches an identifier in the drug holder data. Accordingly, the established dosing parameters are those that are relevant for delivery of the drug from the drug holder of the drug administration device. In this way, it may be ensured that the drug administration device has the required dosing parameters established for carrying out the administration of the drug.

The method can also include communicating the drug holder data from the processor to the server. The relevant subset of dosing parameter data can then be determined on the server. This determination on the server may be an efficient approach for ensuring that only the relevant subset of dosing parameter data is communicated from the server to the processor. This determination on the server may save communication resources such as bandwidth when communicating the relevant subset of data parameter data from the server to the processor.

The method can include communicating drug administration device data from the processor to the server. The drug administration device data is data that is associated with aspects of the drug administration device. In general, the drug administration device data can contain data that relates to capabilities of the drug administration device.

For example, the drug administration device data can relate to the drug administration device's capabilities in relation to a rate at which the drug is administered from the drug administration device. The rate can depend at least in part on the physical construction of the drug administration device, e.g., the device's components may allow for a maximum rate of delivery beyond which one or more of the components may malfunction and/or become disabled for any future drug deliveries.

For another example, the drug administration device data can relate to a dosage size that the drug administration device is capable of administering. The drug holder has a maximum amount of drug that it can hold, which at least in the case of a single-use device defines a maximum possible dose size that can be delivered from the drug administration device. In embodiments in which the drug administration device is a nasal spray device or an inhaler, e.g., the inhaler 300 of FIG. 3, the dosage size is typically a certain set amount (or certain range of amounts) defined by how much drug is released in response to manual depression of, e.g., the nasal spray device's dispensing head (nozzle) or the inhaler's drug holder (canister).

For yet another example, the drug administration device data can specifically identify a form of the drug administration device, e.g., as an autoinjector, as an infusion pump, as an inhaler, as a nasal spray device, etc. In this way, the drug administration device data can differentiate the drug administration device from at least one other form of drug administration device.

The drug administration device data can be utilized by the server in embodiments in which the server determines the relevant subset of dosing parameter data and sends only the relevant subset of dosing parameter data to the processor instead of also sending the non-relevant subset of dosing parameter data to the processor. In this way, the relevant subset of dosing parameter data may be ensured to be appropriate for the drug in the drug holder, based on the drug holder data, as well as for the drug administration device, based on the drug administration device data.

Communicating data between various components in the various embodiments described herein can include encrypted communication. In an exemplary embodiment, the communication between the processor and the server can include encrypted communication, and the communication between the drug holder and the processor can include encrypted communication. The use of encrypted communication increases security and reduces a risk that unauthorized communications may occur. Such unauthorized communications may adversely affect the operation of the drug administration device and so it is beneficial to avoid their occurrence. Possible encryption algorithms that can be utilized include Data Encryption Standard (DES), TripleDES, RSA, Advanced Encryption Standard (AES), and TwoFish.

In an exemplary embodiment, a key-based security system, such as a public key/private key cryptographic system, can be used to allow for data encryption and decryption. Public and private keys can be stored in a memory and can be generated using cryptographic algorithms. Keys can be used to encrypt data for transmission and to decrypt encrypted data received from a different computing device. In such systems, a public key associated with the intended receiver of the data can be utilized to encrypt data, however, only the recipient's private key can be used to decrypt the encrypted data. In at least some embodiments, a cryptographic system such as a public key infrastructure (PKI), in which one or more third parties, known as “certificate authorities,” can be used to certify ownership of the public and private key pairs. Examples of key-based security systems include the Diffie-Hellman key exchange protocol, the Digital Signature Standard (DSS) protocol, password-authenticated key agreement protocols, the Rivest-Shamir-Adelman (RSA) encryption algorithm, the Cramer-Shoup cryptosystem, and the YAK authenticated key agreement protocol.

More particularly, encryption is achieved with algorithms that use a key to encrypt and decrypt messages by turning text or other data into an unrecognizable digital form and then by restoring it to its original form. The longer the key, the more computing is required to crack the code. Computer keys are made of bits of information of various length. For example, an 8-bit key has 256 (2 to the eighth power) possible values. For another example, a 56-bit key creates 72 quadrillion possible combinations. If the key is 128 bits long, or the equivalent of a 16-character message on a personal computer, a brute-force attack would be 4.7 sextillion (4,700,000,000,000,000,000,000) times more difficult than cracking a 56-bit key. With encryption, unauthorized use of the data is generally prevented, even in the rare event that the data transmitted is intercepted by an unauthorized party.

A unique identification (ID) number or code is registered and stored in a memory of a sender device (e.g., the drug administration device, the server, etc.), such as during the manufacturing process before any use thereof. The ID number/code is unique to the sender device, although the ID number/code can include additional identifying information, such as a model or model family number or code, that is common to a plurality of devices and that may be useful in analyzing trends among a plurality of related devices. In an exemplary embodiment, the ID number is transmitted to a recipient with data to facilitate the recipient's identification of the data as coming from that particular device. The recipient can perform this identification in any number of ways, such as by looking up the ID number/code in a lookup table stored at the recipient that correlates ID numbers/codes of sender devices that may communicate with the recipient to particular keys so the recipient can identify which previously generated key to use to decrypt the data received from the sender device. The lookup table can also correlate the ID numbers/codes to particular sender devices. The ID number/code may not be encrypted in the data transmitted from the sender device to the recipient to allow the recipient to read the ID number/code and identify the correct key to use for decryption.

The key generated at the sender device for communication with a particular recipient is shared with the recipient and stored at the recipient in a memory thereof, as discussed above. In some embodiments, instead of the sender device generating the key and transmitting the key to the recipient for storage at the recipient, the recipient can perform the key generation and transmit the key to the sender device for storage thereat. In other embodiments, using certain key generation protocols that will be appreciated by a person skilled in the art, each of the recipient and the sender device participate in key generation such that a key is transmitted to one or the other of the recipient and the sender device, which may help improve security since avoiding key transmission prevents the key from being intercepted by an unauthorized party.

In order to authenticate a source of the various data communicated between elements as described herein, a hash function can be utilized. For example, an intended recipient of data (such as a drug administration device or an external device) can send a random generated number to a source of the data, and then the source of the data (such as a server or a drug holder data storage component) can utilize a hash function to create an encrypted identifier. This encrypted identifier is then sent to the recipient which can verify the authenticity of the communication by decrypting and verifying the identifier. The encrypted identifier can also combine a serial number or batch number in relation to the drug holder data which can be used to further identify the drug holder. The encryption can just be used for a validation term as opposed to encrypting all data in order to save bandwidth, storage size, and/or processor speed.

As mentioned above, the established dosing parameters can be stored in a memory of the drug administration device. In this way, the drug administration device can have the dosing parameters stored locally and be able to utilize the dosing parameters for operation of the drug administration device regardless of communications with the drug holder or the server.

It is possible that in certain embodiments the memory of the drug administration device already has dosing parameters stored therein before the establishment of the dosing parameters, such as by the dosing parameters being pre-programmed therein or as a result of a previous dosing parameters establishment process. In the case of the drug administration device already having dosing parameters stored therein, the established dosing parameters can replace the existing dosing parameters that are stored in the memory and, accordingly, the method can include updating these dosing parameters. This updating may be advantageous if the existing dosing parameters are no longer relevant, either because the relevant dosing parameter data has been updated on the server or because new dosing parameter data is now relevant given a change in the drug holder in the drug administration device.

A method of operating a drug administration device can include the method of establishing dosing parameters for the drug administration device as described herein and also include assessing a suitability of the dosing parameters and, if the dosing parameters are not suitable, preventing dispensing of the drug from the drug administration device, and, if the dosing parameters are suitable, dispensing the drug from the drug administration device based on the dosing parameters. By assessing the suitability of the dosing parameters, it may be possible to ensure that the drug administration device does not attempt to implement dosing parameters that are unsuitable. The dosing parameters may be unsuitable due to the capabilities of the drug administration device being incompatible with the dosing parameters, or the dosing parameters may be unsuitable in relation to possible effects they may have on the patient receiving the drug from the drug administration device. For example, a given patient may have a maximum or minimum dosage volume that is suitable to be administered to them due to any number of reasons such as age, diagnosed condition, etc. This maximum or minimum dosage volume can be different to what would be suitable for a different given patient.

The assessment of the suitability of the dosing parameters can be carried out by the processor. In the event that the dosing parameters are found to not be suitable, the drug administration device can be configured to prevent the dispensing of the drug, such as by using a device operation prevention mechanism described herein. The drug administration device can be further configured to alert a user to the presence of unsuitable dosing parameters, which may allow the user to take corrective action to address the possible current unusability of the drug administration device. The alert can be, e.g., an audible alert and/or a visual alert. If the dosing parameters are considered not to be suitable, the drug administration device can be configured to prevent the unsuitable dosing parameters from being stored in the memory of the drug administration device. In this way, the drug administration device will rely on previously acceptable dosing parameters that are stored in the memory. Thus, the drug administration device can continue operating in a previously acceptable manner.

Assessing the suitability of the dosing parameters can include comparing the dosing parameters with acceptable dosing parameters stored in a memory of the drug administration device or the external device. In this way, it may be ensured that the dosing parameters are in accordance with dosing parameters that are considered acceptable for the drug administration.

In general, an acceptable parameter defines a value (or a range of values) that is indicative of successful delivery of a drug from a drug administration device. The acceptable parameter can be predefined prior to use of the drug administration device, such as by being established by a manufacturer of the drug administration device and/or the drug being delivered by the drug administration device. For example, an acceptable parameter can include a speed of a drug administration device's needle being inserted into a patient when the drug administration device in an injector that includes a needle. Too slow a speed can cause failed needle insertion and thus failed drug delivery. For yet another example, an acceptable parameter can relate to a motor of a drug administration device, such as a speed of the motor or a duration of operation of the motor. Too slow a speed can cause failed drug delivery. Too short a duration of operation of the motor can cause failed drug delivery. For another example, an acceptable parameter can include a flow rate of a drug administered by a drug administration device. Too low a flow rate can cause failed drug delivery. For another example, an acceptable parameter can include a temperature. Too low a temperature can indicate that the drug is likely at a cold enough temperature that its delivery to the patient will be uncomfortable.

In embodiments in which the drug administration data includes authentication data, the dosing parameters can include a parameter for counterfeiting that has only two possible states, a first state indicating no counterfeiting detected and a second state indicating probable counterfeiting detected. The first state can be defined as the acceptable parameter for the parameter for counterfeiting. The parameter for counterfeiting being in the first state allows the drug to be administered in accordance with the parameters of the algorithm that define the dosing regimen. The parameter for counterfeiting being in the second state prevents the drug from being administered from the drug administration device. The parameter for counterfeiting may thus provide a safety mechanism to prevent potentially unsafe drug delivery, e.g., by being in the second state. The drug can be prevented from being administered in accordance with the parameters of the algorithm that define the dosing regimen in a variety of ways, such as by using a device operation prevention mechanism described herein and/or by the processor that executes the algorithm being barred from executing the algorithm if the parameter for counterfeiting is determined to be in the second state.

In an exemplary embodiment, the comparison of a parameter, e.g., a dosing parameter, to an acceptable parameter is performed by the processor, which as discussed above can be the processor of the drug administration device or of the external device. The comparison of each of the dosing parameters with acceptable parameters can be performed in a variety of ways. For example, an acceptable parameter can include a predefined range of values, and the comparing can include determining whether a dosing parameter is within the predefined range of values so as to be indicative of successful drug delivery. For another example, the acceptable parameter can include a predefined threshold value, and the comparing can include determining whether a dosing parameter is above the predefined threshold value so as to be indicative of successful drug delivery. For another example, the acceptable parameter can include a predefined threshold value, and the comparing can include determining whether a dosing parameter is below the predefined threshold value so as to be indicative of successful drug delivery.

The method can include assessing authenticity of the dosing parameter data by comparing authentication data associated with the dosing parameter data with acceptable dosing parameter authentication data. By assessing the authenticity of the dosing parameter data, the possibility of unauthorized dosing parameter data being utilized for forming the dosing regimen may be reduced. The method can additionally or alternatively include assessing authenticity of the drug administration device and/or the drug administration device's drug holder by comparing authentication data associated with the drug administration device and/or the drug administration device's drug holder with acceptable authentication data. By assessing the authenticity of the drug administration device and/or the drug administration device's drug holder, counterfeiting may be detected.

The authentication data can be sent as part of dosing parameter data provided by the server or can be sent alone in embodiments in which authentication data but not dosing parameter data is utilized. The authentication data can be encrypted to increase the security of the authentication data. The comparison of authentication data associated with the dosing parameter data with acceptable dosing parameter authentication data can be carried out by the processor. Similarly, the comparison of authentication data associated with the drug administration device and/or the drug administration device's drug holder with acceptable authentication data can be carried out by the processor. The acceptable dosing parameter authentication data and the acceptable authentication data can be stored on the memory of either the external device or the drug administration device.

The authentication data can have a variety of configurations. For example, the authentication data can specifically identify the drug administration device and/or the drug administration device's drug holder. The authentication data may therefore be useful in identifying the drug administration device as an authorized device containing an authorized drug and/or in identifying the drug administration device's drug holder as an authorized holder containing an authorized drug, thereby helping to ensure that the patient receives the correct drug and that the drug's payer (e.g., the patient, the patient's insurance company, a hospital or other medical care facility at which the drug is administered to the patient, etc.) pays for the correct product. The authentication data can specifically identify the drug administration device and/or the drug administration device's drug holder in a variety of ways, such as with a unique electronic identification (UID) code (numeric and/or alphabetic) that uniquely identifies the drug administration device and/or the drug administration device's drug holder or with invisible ink printed on the drug administration device and/or the drug administration device's drug holder that is not visible by eye but is readable by a light reader.

In at least some embodiments, the authentication data can be used in tracking the drug administration device and/or the drug administration device's drug holder during shipping, which may help identify and/or prevent drug diversion to unauthorized parties. Some drugs may require tracking documentation or be advisable to have tracking documentation of a drug's location throughout the supply chain. For example, tracking esketamine, ketamine, or other controlled substances during the supply chain may be required by a government requirement, e.g., U.S. federal Drug Enforcement Administration requirement, non-U.S. federal requirement, state requirement, or local requirement. For another example, tracking drugs during the supply chain may be an advisable course of action to help prevent diversion and/or evaluate how, when, and/or where diversion occurred. For yet another example, tracking drugs during the supply chain may help ensure that delivery schedules are followed and/or facilitate analysis of inefficiencies in the shipping process.

The authentication data being used during shipping can involve acquiring the authentication data at each planned shipment location during the drug administration device's transport from manufacturer to end site for use and verifying that the drug administration device and/or the drug administration device's drug holder arrived as planned at the planned shipment location. For example, when the authentication data as LTD data is contained as part of an RFID tag, acquiring the LTD code data during shipping can involve the use of an RFID scanner at each planned shipment location during the drug administration device's and/or the drug administration device's drug holder's transport from manufacturer to end site for use. For another example, a bar code scanner can be utilized when the authentication data as UID code data is contained within the form of a bar code. For yet another example, when the authentication data as UID code data is in the form of an integrated circuit, the integrated circuit can be powered and capable of transmitting the UID code data to a receiving communications interface. For still another example, when the authentication data is printed with invisible ink including ultraviolet fluorescent ink, the authentication data can be acquired using an ultraviolet fluorescent light reader. For still another example, when the authentication data is printed with invisible ink including infrared ink, the authentication data can be acquired using an infrared light reader.

In an analogous manner to assessing the authenticity of the dosing parameter data, authenticity can be assessed by comparing authentication data with acceptable authentication data. The authentication data can be encrypted and can be compared with acceptable authentication data by the processor. The acceptable authentication data can be stored in the memory of the external device or the drug administration device. All of the features described in connection with the dosing parameter data can be independently applied to the authentication data. The drug holder authentication data and the dosing parameter authentication data can both be used, or only one of them may be used.

In an exemplary embodiment, a drug administration device configured to use dosing parameters includes a drug holder configured to hold a drug and including a data storage component configured to store drug holder data, a first communications interface configured to receive the drug holder data from the data storage component, a processor configured to receive the drug holder data from the first communications interface, a second communications interface configured to receive at least a relevant subset of dosing parameter data from a server, a memory configured to store dosing parameters, and a dispensing mechanism configured to dispense the drug from the drug holder based on the dosing parameters established by the relevant subset of dosing parameter data. The drug administration device may be able to ensure that it operates with the most appropriate dosing parameters by acquiring the drug holder data and the relevant subset of dosing parameter data.

The drug administration device can be configured to enact any of the features described herein with respect to various drug administration devices (e.g., the drug administration devices of FIGS. 1-3 and 5) and with respect to the method for establishing dosing parameters for a drug administration device described above.

The first and second communications interfaces can be configured for receiving data from the data storage component and the server, respectively. Therefore, each of the first and second communications interfaces is of an appropriate form for interacting with the data storage component and the server, respectively. Where the data storage component and the server rely on the same form of communication, the first communications interface and the second communications interface can be one communications interface.

The memory configured to dosing parameters can include a memory as described herein, e.g., can include ROM, flash memory, one or more varieties of RAM, and/or a combination of memory technologies.

In an exemplary embodiment, a drug administration system configured to use dosing parameters includes a drug administration device that includes a drug holder configured to hold a drug and that includes a dispensing mechanism configured to dispense the drug from the drug holder. The system also includes an external device configured to acquire drug holder data associated with the drug holder. The external device includes a first communications interface configured to receive at least a relevant subset of dosing parameter data from a server, a processor configured to receive the drug holder data, and a memory configured to store dosing parameters. The dispensing mechanism is configured to dispense the drug from the drug holder based on dosing parameters established by the relevant subset of dosing parameter data.

The drug administration system can be configured to enact any of the features described herein with respect to various drug administration devices (e.g., the drug administration devices of FIGS. 1-3 and 5) and with respect to the method for establishing dosing parameters for a drug administration device described above.

The external device is a smart device, e.g., a device that can communicate with other devices via wireless protocols, in an exemplary embodiment. In particular, the external device can be a smart phone, a tablet, a smart watch, or other smart device. The external device can be a dedicated external device for operating as part of the drug administration system. Alternatively, the external device can be a device that has functions apart from its role as part of the drug administration system, which may increase convenience of the system. For example, when the external device is a smart phone, a user's existing smart phone can be used as part of the system.

The external device can include an image sensor configured to acquire the drug holder data. In this way, the image sensor can acquire image data relating to the drug holder. This image data can then be analyzed to extract the required drug holder data. For example, the drug holder data can be in the form of the appearance of the drug holder and thus the image of the drug holder allows the external device to acquire the drug holder data. When the external device is a smart phone or other smart device including camera functionality, the image sensor can be part of the smart device's camera system.

The memory of the external device or the drug administration device can be configured to store a plurality of sets of dosing parameters, e.g., a plurality of dosing regimens. In this way, the memory can store numerous options that can be used for dispensing the drug. The relevant subset of dosing parameter data can indicate which set of the plurality of sets of dosing parameters is adopted for operating the dispensing mechanism. This reduces the amount of data that needs to be acquired from the server and so may increase efficiency of the communications process which may be especially useful when there is poor communications coverage. The dosing parameters in the plurality of sets of dosing parameters can be updated as described herein with respect to establishing dosing parameters. This allows the memory to contain an initial set of dosing parameters which can then be updated as required.

The drug holder data can include an indication of at least one of a type of drug in the drug holder, a concentration of drug in the drug holder, a provenance of drug in the drug holder, and a volume of drug in the drug holder. Therefore, the drug holder data can identify aspects of the drug in the drug holder and so dictate the relevant subset of dosing parameter data accordingly. For example, the drug holder data indicating the type of drug in the drug holder can identify that the relevant subset of dosing parameter data will be the dosing parameter data that is relevant for that drug type. The drug holder data can include indication of multiple aspects of the drug in the drug holder and in this way allow a more precise attainment of dosing parameters that is tailored to the specific drug.

The drug holder data can directly indicate some aspects of the dosage regimen. For example, the drug holder data can dictate a dosage volume, and the dosage volume can be combined with other dosing parameters established by the relevant subset of dosing parameter data.

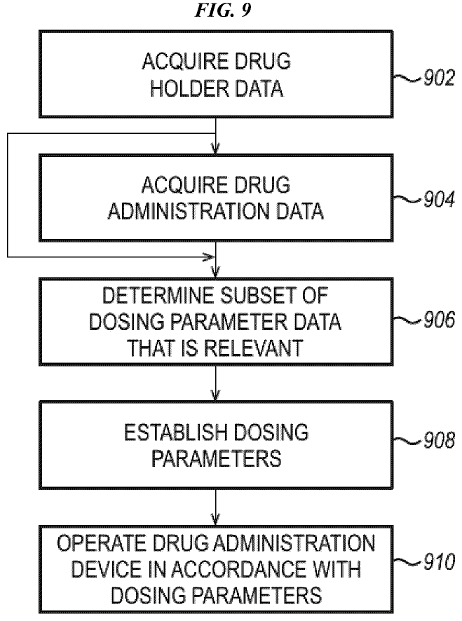

An embodiment of establishing dosing parameters is outlined in FIG. 9. In this embodiment, drug holder data is acquired 902. Optionally, drug administration data is also acquired 904. After acquiring the drug holder data, and optionally the drug administration data, dosing parameter data and, if acquired, the optional drug administration data, that is relevant for the drug holder data can be determined 906. This determination allow for the dosing parameter data to be dosing parameter data that is tailored for given aspects of the drug holder and/or the drug therein, such as drug type, drug concentration, and volume of drug in the drug holder, as well as that is relevant for the drug administration device's capabilities as indicated by the drug administration data (if acquired and used), such as dosing volumes and dosing rate.

In embodiments including authentication data, the authentication data can always be considered relevant data to help ensure that counterfeit protection is always provided, e.g., that the processor compares the received authentication data with data in a lookup table of authorized codes to determine authenticity, such as whether the drug administration device and/or the drug administration device's drug holder is among the authorized devices (code included in the lookup table to indicate not counterfeit) or is not among the authorized devices (code not included in the lookup table to indicate probable counterfeiting).

After determining the relevant subset of dosing parameter data, the dosing parameters can be established 908. As discussed above, the dosing parameters can be directly indicated by the dosing parameter data. In other words, the dosing parameter data can dictate the dosing parameters to be used, such as dosage volume, dosage frequency, and dosage time. Alternatively, the dosing parameter data can indicate which set of dosing parameters that are already present on the drug administration device should be used. The established dosing parameters can be stored in a memory associated with the drug administration device. After establishing the dosing parameters, the drug administration device can be operated in accordance with the established dosing parameters 910. As mentioned above, in embodiments including authentication data that specifically identifies the drug administration device and/or the drug administration device's drug holder, the parameter for counterfeiting being in the first state allows the drug to be administered in accordance with the parameters of the algorithm that define the dosing regimen, and the parameter for counterfeiting being in the second state prevents the drug from being administered from the drug administration device.

In embodiments including authentication data, printed authentication data may also be used to provide further counterfeit protection. Alternatively, printed authentication data can be used to provide counterfeit protection instead of electronic authentication data.

The printed authentication data can include overt printed authentication data printed on the drug administration device and/or on the drug holder and/or can include covert printed authentication data printed on the drug administration device and/or on the drug holder. In an exemplary embodiment, the printed authentication data includes overt and covert printed authentication data.

The overt printed authentication data includes data printed on the drug administration device and/or on the drug holder that is configured to be visible by eye, e.g., by a user looking at the drug administration device and/or the drug holder. The overt printed authentication data is configured to allow the user to determine authenticity of the drug administration device and/or the drug holder by visibly confirming that the overt printed authentication data is present and is in the expected form, e.g., includes the expected words, the expected logo, the expected brand name, etc. The expected form of the overt printed authentication data can be provided to the user in any of a variety of ways, such as by being included in the drug administration device's Instructions For Use (IFU). If the overt printed authentication data is not present and/or is not in the expected form, the user can determine that the drug administration device and/or the drug holder may be counterfeit and should not be used.

The covert printed authentication data includes data printed on the drug administration device and/or on the drug holder that is configured to not be visible by eye and to be visible using a light reader. For example, the covert printed authentication data can be printed using ultraviolet fluorescent ink invisible to the eye but visible to an ultraviolet fluorescent light reader. For another example, the covert printed authentication data can be printed using infrared ink invisible to the eye but visible to an infrared light reader.

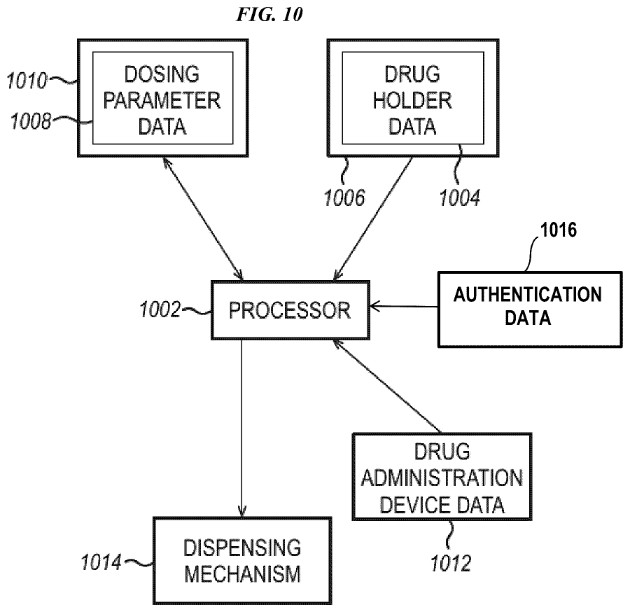

FIG. 10 is a schematic representation of an embodiment of a flow of data associated with a processor 1002 of either a drug administration device or an external device. The processor 1002 receives drug holder data 1004 associated with a drug administration device's drug holder 1006 from the drug holder 1006.

Dosing parameter data 1008 associated with a server 1010 is communicated to the processor 1002 from the server 1010. The processor 1002 can then determine a relevant subset of dosing parameter data based on the drug holder data 1004 that has been communicated to the processor 1002. Alternatively, the processor 1002 can communicate the drug holder data 1004 to the server 1010, and the determination of the relevant dosing parameter data can be carried out on the server 1010 and only the relevant subset of dosing parameter data can then be transmitted from the server 1010 to the processor 1002.

Authentication data 1016 indicative of authenticity of the drug administration device and/or the drug holder 1006 is communicated to the processor 1002 from the drug administration device.

As mentioned above, in some embodiments, dosing parameter data 1008 but not authentication data 1016 is provided to the processor 1002. In other embodiments, authentication data 1016 but not dosing parameter data 1008 is provided to the processor 1002. In still other embodiments, authentication data 1016 and dosing parameter data 1008 are provided to the processor 1002.

Drug administration device data 1012 can also be communicated to the processor 1002 from the drug administration device or the external device. The drug administration device data 1012 can then be used in the determination of the relevant dosing parameter data on the processor 1002. Alternatively, the drug administration device data 1012 can be transmitted from the processor 1002 to the server 1010 when the determination of the relevant dosing parameter data is carried out on the server 1010. Ultimately, the processor 1002 receives from the server 1010 or determines locally the relevant dosing parameter data and utilizes the relevant dosing parameter data to control the drug administration device's dispensing mechanism 1014.

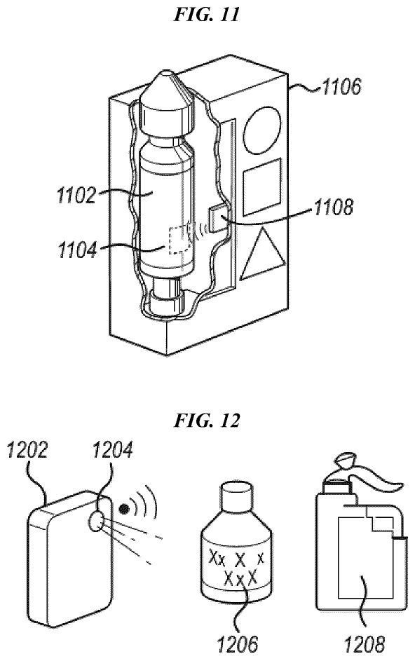

FIG. 11 depicts an embodiment of a drug administration device 1006 that can be used in acquiring drug holder data and/or authentication data. The drug administration device 1006 includes a drug holder 1102 that includes a data storage component 1104 in the form of an RFID tag. The drug administration device 1106 also includes a communications interface 1108 in the form of an RFID scanner. When the drug holder 1102 is in position in the drug administration device 1106, the scanner 1108 is able to receive the drug holder data and/or the authentication data stored in the data storage component 1104. This received drug holder data and/or authentication data can then be communicated as described herein to a processor that is either at the drug administration device 1106 or at an external device.

FIG. 12 depicts an embodiment of a drug administration system that can be used in acquiring drug holder data and/or authentication data. The drug administration system includes an external device in the form of a smart device 1202, a smart phone in this illustrated embodiment. The smart device 1202 includes an image sensor 1204, for example a camera of the smart device 1202. The image sensor 1204 is configured to image a drug holder 1206. By the smart device 1202, e.g., a processor thereof, analyzing the image of the drug holder 1206, drug holder data and/or authentication data can be acquired. This drug holder data and/or authentication data can be communicated to an external server by the smart device 1202 to determine the relevant dosing parameter data at the server using the drug holder data and/or to determine the authenticity of the drug holder 1206, or the drug holder data and/or the authentication data can be utilized by a processor of the smart device 1202 to determine the relevant dosing parameter data using the drug holder data and/or to determine the authenticity of the drug holder 1206. In a similar manner, the smart device 1202 can be configured to image a drug administration device 1208 that is configured to receive the drug holder 1206 and so acquire drug administration device data and/or authentication data. This drug administration device data can then be utilized for determining the relevant dosing parameter data, and/or this authentication data can be used to determine the authenticity of the drug administration device 1208.

### Establishing Compatibility Of Components

In general, establishing compatibility of components relates to determining whether the components are predetermined to be suitable for use with a drug administration device in a particular situation. The establishment of compatibility may help ensure that the drug administration device can function properly to deliver a drug to a patient and/or help ensure that the patient is not injured or otherwise harmed by use of a drug administration device that includes a component that is incompatible therewith and should not be used with the drug administration device.

In an exemplary embodiment, a method of establishing compatibility of components of a drug administration device includes acquiring first component data relating to a first component of the drug administration device, comparing the first component data with acceptable first component data, and setting an operational status of the drug administration device based on the comparison of first component data with acceptable first component data.

The method of establishing compatibility of components of the drug administration device may ensure that the correct first component is utilized with the drug administration device. This may reduce a risk of inadvertently using unsuitable components that may lead to malfunction of the drug administration device and/or incorrect administration of the drug, which may be dangerous for the patient receiving the drug. An example of an unsuitable component is a drug within a drug holder that is not suitable for use with the drug administration device.

The method can establish the compatibility of a single component of the drug administration device or can establish the compatibility of a plurality of components of the drug administration device. Examples of components for which compatibility can be established include a drug holder, a drug within the drug holder, syringe type (for drug administration devices that include a syringe), needle type (for drug administration devices that include a needle), catheter type (for drug administration devices that include a catheter), and sensor type (for drug administration devices that include a sensor).

Acquiring the first component data can include communicating the first component data from the component to an external device. Alternatively, acquiring the first component data can include communicating the first component data from the component to another component, such as a processor, of the drug administration device. The processor can be the same processor used for comparing the first component data with the acceptable first component data and for setting the operational status of the drug administration device, or the processor can be a different processor.

Communicating the first component data can include communicating the first component data from a data storage component. The first component can include the data storage component. As discussed above, examples of data storage components are an integrated circuit, an RFID tag, and a bar code. The first component data can be stored utilizing a single data storage component or a plurality of data storage components. If a plurality of data storage components are used, each can be different from one another, which may help provide redundancy and/or allow for first component data retrieval even if a certain type of data communication is currently unavailable, e.g., if an RFID scanner is absent or damaged. As also discussed above, acquiring data (e.g., the first component data) from the data storage component requires the use of an appropriate communications interface for receiving the drug holder data communicated from the drug holder, such as an RFID scanner, a bar code scanner, or integrated circuitry. In embodiments in which compatibility of components is established and dosing parameters are established and the data storage component that contains the first component data is the data storage component or has the same form as the data storage component for storing the drug holder data, the same reader may be used for obtaining both sets of data.

The details described herein in relation to the first component and the first component data are equally relevant to any further components and their respective component data. Various forms of component data storage and acquisition means may be utilized in combination, as appropriate.

Comparing the first component data with the acceptable first component data can utilize a processor. The processor can be part of the drug administration device or can be part of an external device. The acceptable first component data can be stored in a memory. The memory can be part of the drug administration device or can be part of the external device. When the processor is part of the drug administration device it is preferable that the memory is part of the drug administration device as well to avoid the need to communicate with an off-board memory. Similarly, when the processor is part of the external device, it is preferable that the memory is part of the external device as well to avoid the need to communicate with an off-board memory.

Comparing the first component data with the acceptable first component data can include comparing first component parameter(s) of the first component data with acceptable parameter(s) of the acceptable first component data. This comparison can occur similar to that discussed above regarding the comparison of the dosing parameters with acceptable dosing parameters. The comparison can including determining whether each of the first component parameters in the first component data matches a corresponding parameter in the acceptable first component data, with a match indicating compatibility and a mismatch indicating incompatibility.

The acceptable first component data can be updatable. Therefore, the suitability of the drug administration device with various components can be updated based on developments in relation to the components and the drug administration device. The drug administration device can include a communications interface configured to receive the updated acceptable first component data and store the updated acceptable first component data in the memory.

As mentioned above, setting the operational status of the drug administration device can be based on the comparison of the first component data with acceptable first component data. Setting the operational status can include maintaining the operational status of the drug administration device if the first component data is determined to correspond with the acceptable first component data. When the first component data is determined not to correspond to the acceptable first component data, the operational status of the drug administration device can be changed from the drug administration device being operational for drug delivery to not being operational for drug delivery (or can remain as being not operational for drug delivery if already set to not being operational for drug delivery). Alternatively, the operational status of the drug administration device can be changed from the drug administration device not being operational for drug delivery to being operational for drug delivery (or can remain as being operational for drug delivery if already set to operational for drug delivery) when the first component data corresponds to the acceptable first component data. The adopted approach can be dictated based on whether a default operational status of the drug administration device is to be the operational status or to be an inhibited operational status in which drug delivery is not possible but other operation(s) may be possible, such as comparing first component data with acceptable first component data, providing user notification(s) on a user interface of the drug administration device, etc. For example, if the default operational status is the inhibited operational status, e.g., where the administration of the drug is prevented but other operation(s) are possible, the operational status can be changed to be the operational status when the first component data is determined to corresponds to the acceptable first component data and any other component data (e.g., second component data for a second component, third component data for a third component, etc.) that may be assessed corresponds to its acceptable component data.

Acquiring the first component data can be carried out on the external device. The external device utilized as part of the first component data comparison can be the same external device that is used as part of establishing the dosing parameters.

As discussed above, the external device may be a smart device. The external device can be a device dedicated to the function of establishing the compatibility of the components of the drug administration device. Alternatively, the external device can be a device that performs other functions, such as a smart phone, a tablet, a smart watch, etc. This multi-functional capability enables a user of the drug administration device to utilize an existing device for establishing the compatibility of components of the drug administration device, which may make checking for compatibility more likely to occur properly and without delay since users are already in the habit of keeping such an external device on their person or otherwise nearby for fast accessibility.

Comparing the first component data with the acceptable first component data can occur on the external device, which may be particularly efficient when the external device acquires the first component data.

Comparing the first component data with the acceptable first component data can utilize a processor of the drug administration device, e.g., processor that is part of the first component. Using a processor of the drug administration device may be particularly efficient when the first component receives the acceptable first component data from another source, such as another component of the drug administration device. In embodiments in which the first component receives the acceptable first component data, the first component can be configured to carry out the comparison on the processor and communicate the determined compatibility to the rest of the drug administration device as needed, e.g., by communicating the compatibility determination to a processor of the drug administration device, thus allowing for control of the operability of the drug administration device based on the compatibility of the first component.

The first component data can include image data. This image data may be acquired by imaging the drug administration device with the external device. As discussed above, the external device can include an image sensor, which may be in the form of a camera or other image acquisition element of a smart device. The use of image data as the first component data allows the user to take an image of the drug administration device in order to facilitate assessment of the compatibility of the components. Taking an image is an action that many users will be familiar with, and so this approach will not be considered onerous by most users.

When the first component data includes image data, the comparison of the first component data with the acceptable first component data can include assessing the image data for particular markers or patterns representative of one or more particular components. For example, the assessment of the image data can include the processor extracting particular markers or patterns on an external surface of each of the component(s) to assess whether it is a compatible component. In this case, the user can be instructed to image the drug administration device or the individual components in such a manner that allows the acquisition of this marker or pattern data. In order to assist with this, the marker or pattern data can be present at a plurality of locations of the drug administration device and/or the various components of interest.

Acquiring the first component data can include communicating the first component data from the first component to the processor of the drug administration device.

Setting the operational status of the drug administration device can include flagging that the operational status should be fully operational when the first component data corresponds with the acceptable first component data, and flagging that the operational status should not be fully operational when the first component data does not correspond with the acceptable first component data. Flagging the operational status can include writing data to a memory e.g., the memory of the drug administration device or the external device, that indicates that the operational status should be fully operational (or not be fully operational) based on the comparison of the first component data with the acceptable first component data. This flag data can then be read as needed in assessing the compatibility of components.

The flagging in relation to the first component data can be incorporated into a wider approach that involves a plurality of flags that are each set to indicate whether the operational status should be fully operational based on whether certain criteria are fulfilled. For example, one or more additional flags can be associated with the compatibility of components beyond the first component. In this way, setting the operational status of the drug administration device can include setting the operational status as fully operational when all of the flags (the flag for the first component and the one or more additional flags) indicate that the operational status should be fully operational and setting the operational status as not fully operational when any of the flags (the flag for the first component and the one or more additional flags) indicates that the operational status should not be fully operational. This multi-flag approach allows the operational status to be set based on a range of different criteria and thus effectively operates as a check list when required for an assessment of whether the drug administration device should be operational for drug delivery or not. For example, setting the operational status of the drug administration device can include flagging that the operational status should be fully operational for drug delivery only when the first component data corresponds with the acceptable first component data, and when second component data corresponds with acceptable second component data, and so on in relation to one or more additional components of interest.

Although it is described that the flags can be used to assess whether the drug administration device should be fully operational for drug delivery, the presence of the flags can be used in setting various variable parameters of the drug administration device as appropriate, e.g., in setting variable parameters of a dosing algorithm defined by a dosing regimen. Therefore, the operational status can be set appropriately in view of the flags that are present. As noted above, these flags can be in the form of data (so-called “flag data”) that is written to memory. This flag data can then be read for use in assessing whether the drug administration device should be fully operational.

In an exemplary embodiment, the first component is any component of the drug administration device that is configured to be removable and replaceable. For example, the first component can be a drug holder of the drug administration device when the drug holder is configured to be removably and replaceably received by the drug administration device. The ability to check the compatibility of the drug holder ensures that the drug holder is suitable for use with the drug administration device. Ensures that the drug holder is suitable for use with the drug administration device may ensure that none of an incorrect drug, incorrect drug concentration, and incorrect dosage form is inadvertently used with the drug administration device, which may improve safety and outcomes for the patient. The compatibility check of the drug holder can be conducted as part of the process of establishing the dosing parameters described herein. In particular, the drug holder data can be used as at least part of the first component data used in the compatibility assessment.

When the first component data relates to a drug holder, the first component data can include an indication of at least one of a type of drug in the drug holder, a provenance of the drug, a concentration of drug in the drug holder, an expiry date of the drug, and a volume of drug in the drug holder. This first component data can be indicative of whether the drug holder is compatible with the drug administration device.

When the first component data relates to feature(s) of the first component itself, then the comparison of the first component data with the acceptable first component data can be made with known acceptable forms of the first component. For example, the first component data can convey a type of drug in a drug holder, and the comparison of the first component data and the acceptable first component data can include comparing the type of drug of the drug holder with known acceptable drug type(s) for the drug administration device (e.g., as indicated in a lookup table stored in a memory accessible to the processor executing the comparison), and thus allow for setting the operational status of the drug administration device accordingly.

The first component data can include an indication of compatible drug dispensing mechanism parameters. In this way, the first component data can indicate what one or more forms of drug dispensing mechanisms are suitable for operating with the first component. Therefore, the first component data can convey acceptable form(s) of drug dispensing mechanisms that can be used with the first component.

The first component can include a dispensing mechanism, and the first component data can include an indication of one or more capabilities of the drug dispensing mechanism. In this way, the compatibility assessment of the first component data with the acceptable first component data can be based on whether the one or more capabilities of the drug dispensing mechanism are acceptable for use with the other components of the drug administration device, for example a drug holder.

The first component data can include an indication of compatible drug holder parameters. In this way, the first component data can directly indicate type(s) of drug holders that can be used with the drug administration device.

The first component data can be encoded on the first component. In other words, the first component data can be present on the first component. This encoding on the first component may ensure that the first component has the first component data readily accessible and may reduce a risk that the first component data becomes separated from the first component and thus be unavailable for access for assessing compatibility of the first component with the drug administration device.

In an exemplary embodiment in which compatibility of components of a drug administration device are established, a drug administration device includes a housing, a drug holder configured to hold the drug, and being at least partially within the housing, a dispensing mechanism configured to dispense the drug and being at least partially within the housing, data storage configured to store first component data, and a processor configured to receive and compare the first component data with acceptable first component data and set an operational status of the drug administration device based on the comparison.

In another exemplary embodiment in which compatibility of components of a drug administration device are established, a drug administration system includes a drug administration device that includes a housing, a drug holder configured to hold the drug and being at least partially within the housing, and a dispensing mechanism configured to dispense a drug and being at least partially within the housing. The drug administration system also includes an external device configured to acquire first component data relating to a first component of the drug administration device. The external device includes a processor configured to receive and compare the first component data with acceptable first component data and configured to set an operational status of the drug administration device based on the comparison.

The drug administration device and the drug administration system of these two exemplary embodiments may enable the compatibility of components to be assessed based on the first component data and for the operational status of the drug administration device to be set accordingly as discussed herein.

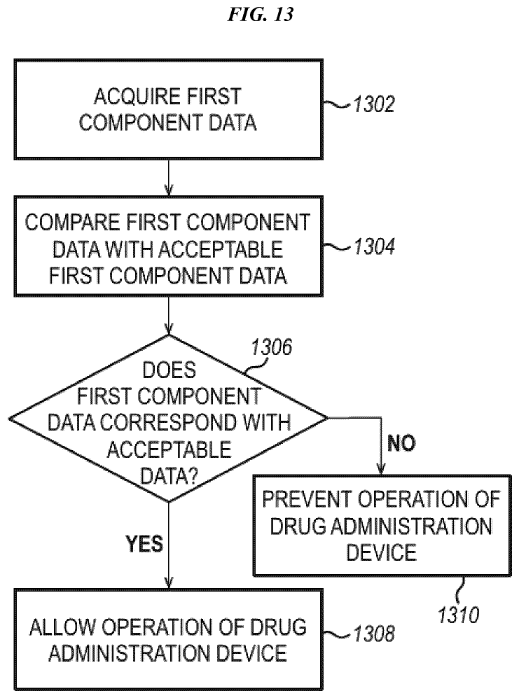

FIG. 13 schematically illustrates an embodiment of a method of establishing compatibility of components. As shown, the first component data is acquired 1302. As noted herein, the acquisition can occur by, e.g., the first component data being communicated from a data storage component or an image being taken of a first component and the first component data being extracted from the image data.

The first component data is then compared 1304 with acceptable first component data. As noted herein, this comparison can be carried out using a processor, e.g., a processor of a drug administration device or of an external device. The acceptable first component data can be stored in a memory associated with the processor, and the processor can compare the acquired first component data with the acceptable first component data present in the memory.

Based on this comparison, the processor determines 1306 whether the first component data corresponds with the acceptable first component data. In the situation that the first component data is determined 1306 to correspond with the acceptable first component data, the processor allows 1308 operation of the drug administration device for drug delivery, such as by writing a flag as a piece of data that indicates that drug delivery operation of the drug administration device can proceed when needed. The drug administration device can then be configured to check the status of the flag prior to any administration of drug. If the first component data is determined 1306 to not correspond with acceptable first component data, then the processor prevents 1310 operation of the drug administration device, such as by removing an existing flag indicating that the drug administration device is operational, not writing a flag as a piece of data that indicates that drug delivery operation of the drug administration device can proceed when needed, or writing a flag as a piece of data that indicates that drug delivery operation of the drug administration device cannot proceed when needed.

As discussed above, the operation of the drug administration device for drug delivery can be controlled using a device operation prevention mechanism that can be controlled by the drug administration device, e.g., by a processor of the drug administration device. Therefore, the device operation prevention mechanism can be placed in a locked state (preventing drug delivery) or an unlocked state (allowing drug delivery) according to the compatibility assessment.

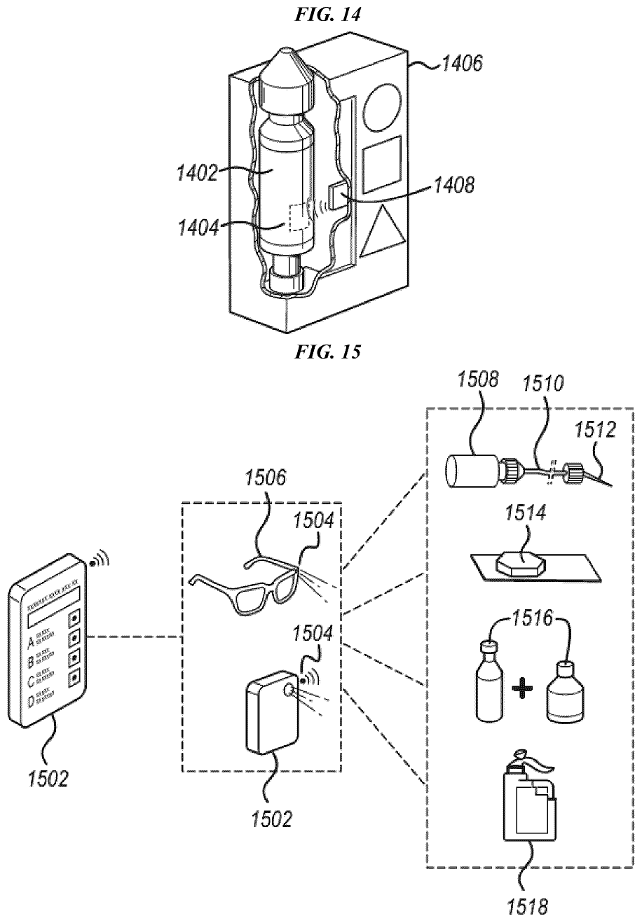

FIG. 14 depicts an embodiment of a drug administration device that can be used in acquiring first component data associated with a first component. In this example, the first component is a drug holder 1402 of the drug administration device, and the first component data is present as an RFID tag 1404 attached to the drug holder 1402. An RFID scanner 1406 is part of the drug administration device's housing 1408 and is positioned to be able to interact with the RFID tag 1404 when the drug holder 1402 is present in the housing 1408. In this way, the scanner 1406 can acquire first component data from the drug holder 1402. This first component data can then be conveyed to a processor (of the drug administration device or of an external device) where the first component data is compared with acceptable first component data as discussed herein, and then the processor sets the operational status of the drug administration device for drug delivery based on this comparison as also discussed herein.

FIG. 15 illustrates an embodiment of a drug administration system including an external device. The external device in this embodiment is in the form of a smart device 1502 configured to wirelessly interact with other components. The external device 1502 is associated with an image sensor 1504 configured to acquire the first component data in the form of image data. The image sensor 1504 can be present on the smart device 1502 as shown as one alternative in FIG. 15. Alternatively, as shown as another alternative in FIG. 15, the image sensor 1504 can be present on another external device, such as glasses 1506 that can be worn by a user.

The image sensor 1504 can be used to acquire image data of a range of components associated with a drug administration device and thus confirm whether all the imaged components are appropriate. The imaged components can include any one or more of a syringe 1508 of a first drug administration device, a catheter 1510 of the first drug administration device, a needle 1512 of the first drug administration device, a sensor 1514 (for example a glucose sensor) associated with the first drug administration device, drug holders 1516 each configured to be inserted into a housing 1518 of a second drug administration device, and a housing 1518 of the second drug administration device.

As noted herein, establishing compatibility of components can determine whether the components that form a drug administration device are appropriate. The outcome of the assessment can be conveyed to a user via an alert using a device indicator and/or a user interface. The alert can be a visual alert on a screen associated with the drug administration device and/or can be an audio alert. The alert can be different for an indication that the components are compatible and administration can proceed compared to an alert that indicates there is a problem with compatibility and so administration cannot proceed. Alternatively, an alert may only be issued when compatibility is problematic or only when no compatibility issues have been identified.

When a drug administration device has stored dosing parameter data, establishing compatibility of components may ensure compatibility with the dosing parameter data. For example, if the drug administration device has dosing parameter data that indicates a daily frequency of drug administration, establishing compatibility of components may ensure that the components are compatible with the daily dosing regimen. The dosing parameter data may be stored in a memory of the drug administration device or of an external device, and the comparison of whether the daily dosing regimen is suitable given the first component data or other component data can be carried out by the processor.

In addition to assessing the compatibility of the components, other approaches can be used for ensuring that only compatible devices are utilized. For example, a physical interface between the components can be sized and shaped to limit the physical compatibility to include components that are known to be compatible. In this way, the number of components that are able to be connected to form the drug administration device is restricted and thereby reduces the possibility of utilizing non-compatible components.

All of the devices and systems disclosed herein can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It can be preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device including internal circuitry is described in more detail in U.S. Pat. Pub. No. 2009/0202387 published Aug. 13, 2009 and entitled “System And Method Of Sterilizing An Implantable Medical Device.” It is preferred that device, if implanted, is hermetically sealed. This can be done by any number of ways known to those skilled in the art.

The present disclosure has been described above by way of example only within the context of the overall disclosure provided herein. It will be appreciated that modifications within the spirit and scope of the claims may be made without departing from the overall scope of the present disclosure.

1-59. (canceled)

60. A drug administration system, comprising:
a drug administration device configured to removably and replaceably receive a drug holder therein such that the drug administration device is configured to receive therein each of a plurality of drug holders, the drug administration device comprising:
a memory,
a communications interface configured to, with a first one of the plurality of drug holders received in the drug administration device, acquire data from a data storage component of the first one of the plurality of drug holders received in the drug administration device, and
a processor configured to, with the first one of the plurality of drug holders received in the drug administration device:
establish a dosing parameter based on the data acquired from the data storage component,
cause the established dosing parameter to be stored in the memory, and
control drug delivery from the drug administration device based on the established dosing parameter.

61. The system of claim 60, wherein the dosing parameter comprises at least one of a volume of drug to be dispensed, a frequency of drug dispensing, a rate of drug dispensing, a time for the drug to be dispensed, and a temperature of the drug at which the drug should be dispensed.

62. The system of claim 60, wherein the data comprises at least one of an indication of a type of the drug held in the first one of the plurality of drug holders received in the drug administration device, a concentration of the drug held in the first one of the plurality of drug holders received in the drug administration device, a provenance of the drug held in the first one of the plurality of drug holders received in the drug administration device, an expiry date of the drug held in the first one of the plurality of drug holders received in the drug administration device, and a volume of the drug held in the first one of the plurality of drug holders received in the drug administration device.

63. The system of claim 60, wherein the data comprises a volume of the drug; and
controlling drug delivery from the drug administration device based on the parameter comprises dispensing, using a dispensing mechanism of the drug administration device configured to dispense the drug from the first one of the plurality of drug holders received in the drug administration device, based on the established dosing parameter.

64. The system of claim 60, wherein the data comprises at least one of a provenance of the drug held in the first one of the plurality of drug holders received in the drug administration device and an expiry date of the drug held in the first one of the plurality of drug holders received in the drug administration device; and
controlling delivery of the drug from the drug administration device based on the parameter comprises preventing the drug held in the first one of the plurality of drug holders from being delivered from the drug administration device.

65. The system of claim 60, wherein the data storage component is a code and the communications interface is a code reader.

66. The system of claim 60, wherein the data storage component is a tag and the communications interface is a tag scanner.

67. The system of claim 60, wherein the drug administration device is a pump.

68. The system of claim 60, wherein the drug administration device is one of a nasal spray device and an inhaler.

69. A method of establishing dosing parameters for a drug administration device, the method comprising:
the communications interface of claim 60 acquiring the data from the first one of the plurality of drug holders received in the drug administration device of claim 60; and
the processor of claim 60, with the first one of the plurality of drug holders received in the drug administration device:
establishing the dosing parameter based on the data acquired from the data storage component,
causing the established dosing parameter to be stored in the memory of claim 60, and
controlling drug delivery from the drug administration device of claim 60 based on the established dosing parameter.

70. A drug administration system, comprising:
a drug holder configured to hold a drug, wherein the drug holder comprises a data storage component configured to store data regarding at least one of the drug and the drug holder; and
a pump configured to operably couple to the drug holder, the pump comprising:
a memory,
a communications interface configured to, with the drug holder operably coupled to the pump, acquire the data from the data storage component, and
a processor configured to
establish a dosing parameter based on the data acquired from the data storage component,
cause the established dosing parameter to be stored in the memory, and
control delivery of the drug from the drug holder operably coupled to the pump based on the established dosing parameter.

71. The system of claim 70, wherein the dosing parameter comprises at least one of a volume of drug to be dispensed, a frequency of drug dispensing, a rate of drug dispensing, a time for the drug to be dispensed, and a temperature of the drug at which the drug should be dispensed.

72. The system of claim 70, wherein the data comprises at least one of an indication of a type of the drug held in the drug holder, a concentration of the drug held in the drug holder, a provenance of the drug held in the drug holder, an expiry date of the drug held in the drug holder, and a volume of the drug held in the drug holder.

73. A method of establishing dosing parameters for a drug administration device, the method comprising:
the communications interface of claim 70, acquiring the data from the drug holder of claim 70; and
the processor of claim 70:
establishing the dosing parameter based on the data acquired from the data storage component,
causing the established dosing parameter to be stored in the memory of claim 70, and
controlling delivery of the drug from the pump of claim 70 based on the established dosing parameter.

74. A drug administration system, comprising:
a drug administration device configured to removably and replaceably receive a drug holder therein such that the drug administration device is configured to receive therein each of a plurality of drug holders, the drug administration device comprising:
a memory configured to store acceptable component data and to store an operational status of the drug administration device,
a communications interface configured to, with the drug holder received in the drug administration device, acquire component data from a data storage component of the drug holder, and
a processor configured to, with the drug holder received in the drug administration device:
compare the component data acquired from the data storage component with the acceptable component data,
set the operational status based on the comparison, and
control operation of the drug administration device based on the set operational status.

75. The system of claim 74, wherein the component data comprises an indication of at least one of:
a type of the drug held in the drug holder received in the drug administration device,
a provenance of the drug held in the drug holder received in the drug administration device,
a concentration of the drug held in the drug holder received in the drug administration device,
an expiry date of the drug held in the drug holder received in the drug administration device,
a volume of the drug held in the drug holder received in the drug administration device, and
a type of the drug holder received in the drug administration device.

76. The system of claim 74, wherein the operational status is either a first status, which allows the drug held in the drug holder received in the drug administration device to be delivered from the drug administration device, or a second status, which does not allow the drug held in the drug holder received in the drug administration device to be delivered from the drug administration device.

77. The system of claim 74, wherein the component data relates to the drug held in the drug holder received in the drug administration device; and
the comparison determines whether the drug held in the drug holder received in the drug administration device is compatible with the drug administration device.

78. The system of claim 74, wherein the component data relates to the drug holder received in the drug administration device; and
the comparison determines whether the drug holder received in the drug administration device is compatible with the drug administration device.

79. A method of establishing compatibility of components of a drug administration device, the method comprising:
the communications interface of claim 74, acquiring the component data from the drug holder received in the drug administration device of claim 74; and
the processor of claim 74, with the drug holder received in the drug administration device:
comparing the component data acquired from the data storage component with the acceptable component data,
setting the operational status based on the comparison, and
controlling operation of the drug administration device based on the set operational status.
